(19) United States
(12) Patent Application Publication
(10) Pub. No.: US 2009/0304803 A1
(43) Pub. Date: Dec. 10, 2009

(54) Compositions and methods relating to target-specific photodynamic therapy

(75) Inventor: Tayyaba Hasan, Arlington, MA (US)

Correspondence Address: EWARDS ANGELL PALMER & DODGE LLP, P.O. BOX 55874, BOSTON, MA, 02205, US

(73) Assignee: The General Hospital Corporation, Boston, MA (US)

(21) Appl. No.: 11/921,597

(22) PCT Filed: Jun. 6, 2006
(86) PCT No.: PCT/US06/22081

§ 371 Date: Jun. 25, 2009

Related U.S. Application Data
(60) Provisional application No. 60/687,800, filed on Jun. 6, 2005.

(51) Int. Cl. A61K 9/14; A61P 35/00

(52) U.S. Cl. 424/497

(57) ABSTRACT

The invention generally provides methods and compositions useful for providing photodynamic therapy to specific cells or tissues.

### CROSS-REFERENCE TO RELATED APPLICATION AND INCORPORATION BY REFERENCE

This application claims the benefit of the following U.S. Provisional Application No. 60/687,800, filed on Jun. 6, 2005, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by the following grants from the National Institutes of Health, Grant No: R01-CA119388. The government may have certain rights in the invention.

### BACKGROUND OF THE INVENTION

Photodynamic therapy (PDT) is an emerging modality in use for cancer and non-cancer therapeutics. PDT works by using light-mediated cytotoxicity to eliminate undesired cells. It is based on the observation that certain non-toxic chemicals, called photosensitizers, accumulate preferentially in malignant tissues. Therapy involves delivering visible light of the appropriate wavelength to excite the photosensitizer molecule within the cell to the excited singlet state. Following this molecular excitation, there are typically two major photochemical pathways involved in cytotoxicity, and classified somewhat arbitrarily as Type I (direct reactions from the PS excited state with biological substrates) and Type II, via singlet oxygen (¹O₂). Current treatment modalities, which rely on photodynamic therapy for the elimination of undesired cells, could be improved if more specific targeting methods were available. Such methods would selectively target cytotoxicity to undesired cells, while sparing healthy cells and tissues.

### SUMMARY OF THE INVENTION

As will be described in more detail below, the invention provides methods and compositions useful for targeting photodynamic therapy to specific cells or tissues.

In general, the invention features a nanoparticle containing a polymer shell, a photosensitizer core, and an aptamer affixed to the shell. In one embodiment the aptamer binds to an ErbB family member (e.g., ErbB3). In other embodiments, the photosensitizer is a porphyrin (e.g., any one or any combination of the following: a porfimer sodium, hematoporphyrin IX, hematoporphyrin ester, dihematoporphyrin ester, synthetic diporphyrin, O-substituted tetraphenyl porphyrin, 3,1-meso tetrakis porphyrin, hydroporphyrin, benzoporphyrin derivative, benzoporphyrin monoacid derivative, monoacid ring derivative, tetracyanoethylene adduct of benzoporphyrin, dimethyl acetylenedicarboxylate adduct of benzoporphyrin, -aminolevulinic acid, benzonaphthoporphyrazine, naturally occurring porphyrin, ALA-induced protoporphyrin IX, synthetic dichlorin, bacteriochlorin tetra(hydroxyphenyl) porphyrin, purpurin, octaethylpurpurin derivative, etiopurpurin, tin-etio-purpurin, porphycene, chlorin, chlorin e6, mono-1-aspartyl derivative of chlorin e6, di-1-aspartyl derivative of chlorin e6, tin(IV) chlorin e6, meta-tetrahydroxyphenylchlorin, chlorin e6 monoethylendiamine monamide, verdin, zinc methyl pyroverdin, copro II verdin trimethyl ester, deuteroverdin methyl ester, pheophorbide derivative, pyropheophorbide, texaphyrin, lutetium (III) texaphyrin, and gadolinium(III) texaphyrin); a photoactive dye, (e.g., any one or more of the following: merocyanine, phthalocyanine, chloroaluminum phthalocyanine, sulfonated aluminum PC, ring-substituted cationic PC, sulfonated AIPc, disulfonated or tetrasulfonated derivative, sulfonated aluminum naphthalocyanine, naphthalocyanine, tetracyanoethylene adduct, nile blue, crystal violet, azure chloride, rose bengal, benzophenothiazinium, phenothiazine derivative and rose bengal methylene blue); or is any one or more of a Diels-Alder adduct, dimethyl acetylene dicarboxylate adduct, anthracenedione, anthrapyrazole, aminoanthraquinone, phenoxazine dye, chalcogenapyrylium dye, cationic selena, tellurapyrylium derivative, cationic imminium salt and tetracycline.

In various related embodiments the polymer shell contains any one or more of the following polymers: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt. In yet other embodiments the polymer shell contains any one or more of the following polymers: poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), and poly(ethylene terephthalate). In yet other embodiments, the polymer shell contains any one or more of the following polymers: poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate). In one embodiment, the polymer is a PEG-PLGA polymer.

In other related embodiments, the mass of the nanoparticle is about 1-150 KD (e.g., any integer between about 1 and 150, where the bottom of the range is any integer between about 1 and 149, and the top of the range is any integer between about 2 and 150). In one embodiment, the mass of the nanoparticle is about 30-60 KD (e.g., about 30, 35, 40, 45, 50, 55, or 60). In other related embodiments, the size of the nanoparticle is about 1-500 nm (e.g., about 100-400 nm, 200-300 nm, or 10-100) nucleotides, where the bottom of the range is any integer between about 1-499 and the top of the range is any integer between about 2 and 500.

In yet other related embodiments the aptamer includes between about 10-75 (e.g., about 10-30, 30-60, 60-75) nucleotides, where the bottom of the range is any number between about 10 and 74, and the top of the range is any number between about 11 and 75. In another embodiment, the nanoparticle comprises at least two aptamers fixed to its shell, at least one of which binds to ErbB3.

In another aspect, the invention features a pharmaceutical composition for the treatment of a neoplasm, the composition containing an effective amount of the nanoparticle of the previous aspect in a pharmaceutically acceptable excipient. In one embodiment, the composition is suitable for systemic or local delivery. In another embodiment, the composition further comprises a chemotherapeutic (e.g., abiraterone acetate, altretamine, anhydrovinblastine, auristatin, bexarotene, bicalutamide, BMS184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1 -Lproline-t-butylamide, cachectin, cemadotin, chlorambucil, cyclophosphamide, 3′,4′-didehydro4′-deoxy-8′-norvin-caleukoblastine, docetaxol, doxetaxel, cyclophosphamide, carboplatin, carmustine (BCNU),cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermiin, taxol, tretinoin, vinblastine, vincristine, vindesine sulfate, and vinflunine).

In yet another aspect, the invention features a method of producing a phototoxic effect in an undesired cell. The method involves the steps of (a) administering a nanoparticle of the invention to a cell; and (b) administering light to the cell in a dose effective to produce a reactive species, thereby producing a phototoxic effect in the cell. In one embodiment, the cell functions in a disease or disorder (e.g., age-related macular degeneration, an immunoinflammatory disorder, rheumatoid arthritis, a neoplasm, or a pathogen infection). In one embodiment, the method eliminates (i.e., kills) or modulates the cell. In another embodiment, elimination of the cell ameliorates, stabilizes, or treats the disease or disorder.

In another aspect, the invention features a method of reducing the growth or proliferation of a neoplasm in a subject (e.g., a mammal or human patient). The method involves administering a nanoparticle of the invention to a subject diagnosed as having a neoplasm; and administering light to the neoplasm in a dose effective to produce a reactive species, thereby reducing the growth or proliferation of the neoplasm (e.g., an intraperitoneal neoplasm, such as ovarian cancer) in the subject.

In another aspect, the invention features a method of inducing cytotoxicity in a neoplastic cell. The method involves contacting the cell with a nanoparticle of the invention; and administering light to the cell in a dose effective to produce a reactive species, thereby inducing cytotoxicity in the neoplastic cell (e.g., a mammalian cell or a human cell). In various embodiments, the neoplastic cell is in vitro or in vivo. In related embodiments, cytotoxicity is detected by assaying apoptosis (e.g., using a trypan blue exclusion assay).

In yet another aspect, the invention features a method of inducing toxicity in a pathogen. The method involves contacting the pathogen with a nanoparticle of the invention; and administering light to the cell in a dose effective to produce a reactive species, thereby inducing toxicity in the pathogen (e.g., a bacteria, virus, fungi, yeast, protist, or a parasite).

In yet another aspect, the invention features a method of stabilizing, reducing, or ameliorating a pathogen infection in a subject (e.g., a mammal or human patient). The method involves administering the nanoparticle of the invention to a subject diagnosed as having a pathogen infection; and administering light to the site of the infection in a dose effective to produce a reactive species, thereby stabilizing, reducing, or ameliorating the pathogen infection in the subject.

In yet another aspect, the invention features a pharmaceutical composition for the treatment of a pathogen infection, the composition containing an therapeutically effective amount of the nanoparticle of any previous aspect in a pharmaceutically acceptable excipient. In various embodiments, the composition is suitable for systemic or local delivery. In other embodiments, the composition further comprises a second therapeutic (e.g., an antibiotic, a nematicide, a fungicide, a parasiticide, and a biocide).

In various embodiments of any of the above aspects, the pathogen is a bacteria (e.g., Aerobacter, Aeromonas, Acinetobacter, Actinomyces israelli, Agrobacteiuin, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, Corynebacterium diphtheriae, Corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafria, Helicobacter, Klebsiella, Klebsiella pneumoniae, Legionella, Leptospira, Listeria, Morganella, Moraxella, Mycobacteriumn, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia), such as a gram positive bacteria (e.g., Pasteurella species, Staphylococci species, and Streptococcus species) or a gram negative bacteria (e.g., Escherichia coli, Pseudomonas species, and Salmonella species); a virus (e.g., Retroviridae, Picornaviridae, Calciviridae, Togaviridae, Flaviridae, Coronoviridae, Rhabdoviridae, Filoviridae, Paramyxoviridae, Orthomyxoviridae, Bungaviridae, Reoviridae, Birnaviridae; Hepadnaviridae, Parvovirida, Papovaviridae, Adenoviridae, Herpesviridae, Poxviridae, and Iridoviridae), a fungus (e.g., Alternaria, Aspergillus, Basidiobolus, Bipolaris, Blastomyces dermatitidis, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, Candida lusitaniae, Coccidioides, Coccidioides immitis, Cladophialophora, Chlamydia trachiomatis, Candida albicans, Cryptococcus, Cryptococcus neoformans, Cunninghamella, Curvularia, Exophiala, Fonsecaea, Histoplasma, Histoplasma capsulatum, Madurella, Malassezia, Plastomyces, Rhodotorula, Scedosporium, Scopulariopsis, Sporobolomyces, Tinea, and Trichosporon), a protist (e.g., Plasmodium falciparum, Plasmodium malariae, Plasmodium ovale, and Plasmodium vivax), or a parasite (e.g., Babesia microti, Babesia divergens, Leishmania tropica, Leishmania spp., Leishmania braziliensis, Leishmania donovani, Trypanosoma gambiense and Trypanosoma rhlodesiense, Trypanosoma cruzi, Toxoplasma gondii, a filariid, ascarid, capillarid, strongylid, strongyloides, trichostrongyle, or trichurid nematode). In other embodiments of any of the above aspects, the pathogen infection is a nocosmial infection, bacteremia, sepsis, or septic shock.

In yet another aspect, the invention provides kits and pharmaceutical systems.

In one embodiment, the invention provides a kit for producing a phototoxic effect in an undesired cell comprising a nanoparticle of the invention and instructions for use thereof.

In another embodiment, the invention provides a packaged pharmaceutical comprising a nanoparticle and instructions for using said nanoparticle to produce a phototoxic effect in an unwanted cell.

In specific embodiments, the undesired cell is a neoplastic cell or an infected cell and instructions are for using said nanoparticle to treat a neoplasm or infection according to the methods described herein. Other features and advantages of the invention will be apparent from the detailed description, and from the claims.

### BRIEF DESCRIPTION OF THE DRAWINGS

FIGS. 1A-1C show expression of EGFR, ErbB2 and ErbB3 protein and mRNAs in ovarian cancer cell lines. FIG. 1A includes four panels depicting Western blots, which show EGFR, ErbB2 and ErbB3 protein expression in PA-1, OVCAR-5, OVCAR-3, and SKOV-3 ovarian cancer cell lines relative to the house keeping protein, GAPDH. FIGS. 1B and 1C are graphs showing relative levels of EGFR and ErbB3 mRNA expression in the indicated ovarian cancer cell lines.

FIGS. 2A-2F show the cellular localization of free photosensitizer benzoporphyrin derivative (BPD)and PLGA nanoparticles encapsulating the photosensitizer benzoporphyrin derivative (BPD-NP) in OVCAR3 (FIGS. 2A-2C) and PA-1 (2D-2F) cells in media alone (FIGS. 2A and 2D), Media with Free BPD (FIGS. 2B and 2E), and media with BPD-nanoparticles.

FIG. 3 is a graph showing the phototoxicity of free benzoporphyrin derivative (BPD) and PLGA nanoparticles encapsulating the photosensitizer benzoporphyrin derivative (BPD-NP) in three ovarian cancer cell lines.

FIG. 4 provides the Nucleotide sequence of ErbB3 specific and control aptamer. The sequence from 5′ to 3′ is listed for both the specific and the scrambled (control) aptamer.

FIG. 5 is a photomicrograph showing ErbB3-specific aptamer localization in OVCAR3 (target) and PA-1 (non-target) cells.

FIG. 6 is a photograph of a peritoneal view of mouse model of human ovarian cancer. Shown in the photograph are the stomach (s), kidney (k), bifurcating horns of the Uterus (u), Ovary (o), and solid tumor masses (circled). This photograph was taken twenty-eight days after the mouse received an intraperitoneal injection of chemotherapy resistant human ovarian cancer derived from SKOV3 cell line.

FIGS. 7A and 7B show the quantitation of chlorin e6 monoethylendiamine monamide photosensitizer chlorin e6 monoethylendiamine monamide (CMA) in vivo. FIG. 7A is a graph showing the distribution of CMA or CMA-immunoconjugate twenty-four hours after intraperitoneal injection in tumor-bearing mice. FIG. 7B is a graph showing the fluorescence intensity at various times after 5-aminolevulinic acid (ALA) administration.

FIGS. 8A and 8B show that photodynamic therapy with photoimmunoconjugates reduce tumor burden and increase survival of tumor-bearing mice. FIG. 8A is a table showing percentage that conjugation of CMA to an antibody improves treatment efficacy. FIG. 8B is a graph showing that treated mice that received four treatments with a CMA-immunoconjugate and intraperitoneal light irradiation survived significantly longer than untreated control mice.

FIGS. 9A1-9B2 are photomicrographs showing the PCNA staining pattern of tumor samples from mice injected with ovarian cancer cells. FIG. 9A1 shows PCNA staining in untreated tumor from the omentum (9A1) and Lymph nodes (9A2). FIGS. 9B1 and 9B2 shows the combination of photodynamic therapy and C225 in Pelvis (9B1) and Mesentery (9B2) seventy-two hours after the second PDT treatment.

FIG. 10 shows the chemical structure of CMA, which includes a porphyrin ring with an amino group for facilitating conjugation with other agents. CMA, with a molecular weight of 680.71, is soluble in water. To achieve the monomeric form, the solvent includes least 50% organic composition.

FIG. 11 is a graph showing SNAC phototoxicity in prostate cancer cells.

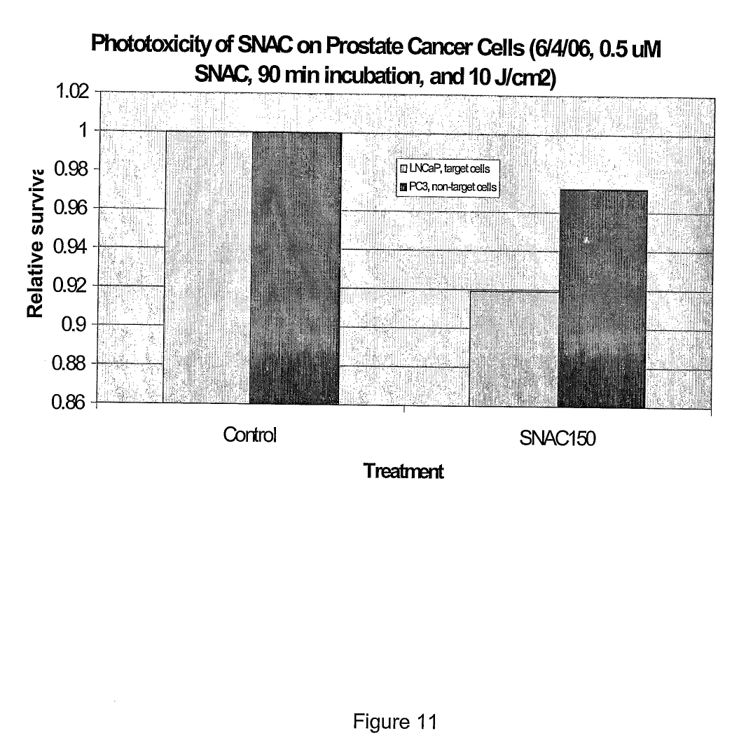

### DETAILED DESCRIPTION OF TIE INVENTION

### Definitions

By “affixed” is meant an association between two substances (e.g, an aptamer and a polymer shell) through covalent or non-covalent bonding (e.g., hydrogen bonding, Van der Waals interactions). Methods of affixing aptamers of the invention include methods of conjugation described throughout this disclosure, and as otherwise known in the art.

By “ameliorate” is meant decrease, suppress, attenuate, diminish, arrest, or stabilize the development or progression of a disease.

By “aptamer” is meant a single-stranded polynucleotide that binds to a cell surface marker (i.e., typically a macromolecule present on the surface of a target cell), such as a receptor, epitope, lipid, carbohydrate, protein or glycoprotein. The aptamer fixed to the polymer shell of the nanoparticle is capable of binding with specificity to a cell surface marker of a target cell. “Binding with specificity” means that nontarget cells are either not specifically bound by the aptamer or are only poorly bound by the aptamer.

The terms “comprises,” “comprising,” “containing” and “having” have the meaning ascribed to them in U.S. Patent law and can mean “includes,” “including,” and the like; “consisting essentially of” or “consists essentially” likewise has the meaning ascribed in U.S. Patent law. Thus, the term is open-ended and allows for the presence of more than that which is recited so long as the basic or novel characteristics of that which is recited is not changed by the presence of more than that which is recited, but excludes prior art embodiments.

By “core” is meant the interior of the polymer shell that consists essentially of at least one photosensitizer. The photosensitizer is encompassed by and in specific embodiments, serves as the relative center of the polymer shell.

By “disease” is meant any condition or disorder that damages or interferes with the normal function of a cell, tissue, or organ. Examples of diseases include cancer or pathogen infection, such as a viral, bacterial, parasite, fungus, or protist infection.

An “effective dose of light” is light provided in a duration, power and wavelength sufficient to produce a reactive species from a photoactivatable compound, such as a photosensitizer.

By “eliminate” a cell is meant to cause cell death.

By “ErbB family member” is meant a receptor expressed on the cell surface having tyrosine kinase activity and structural and/or functional homology to the EGF receptor. Exemplary ErbB family members include the EGF receptor, erbB2, erbB3, and erbB4.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “modulation” is meant any alteration (e.g., increase or decrease) in a biological finction or activity.

By “neoplasm” is meant a disease that is caused by or results in inappropriately high levels of cell division, inappropriately low levels of apoptosis, or both. Cancer is an example of a neoplasm.

By “obtaining” as in “obtaining an agent” is meant synthesizing, purchasing or otherwise acquiring the agent.

By “photodynamic therapy” or “PDT” is meant a treatment that includes the use of light and a light activated chemical compound to stabilize, reduce, or ameliorate a disease or condition that involves the modulation or deletion of an undesired cell.

By “pathogen” is meant any microorganism, such as a bacterium, virus, fungus, protozoan, or parasite, capable of interfering with the normal function of a cell.

By “polynucleotide” is meant is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

By “photosensitizer” is meant a photoactivatable compound, or a biological precursor thereof, that produces a reactive species (e.g., oxygen) having a photochemical (e.g., cross linlcing) or phototoxic effect on a cell, cellular component or biomolecule.

By “polypeptide” is meant any chain of amino acids, regardless of length or post-translational modification.

By “protein” is meant a polypeptide (native or mutant), oligopeptide, peptide, or other amino acid sequence. “Protein” and “polypeptide” are used interchangeably herein without intending to limit the scope of either term.

By “nanoparticle” is meant a photosensitizer core, a polymer shell, and a targeting aptamer affixed to the surface of the shell. A nanoparticle of the invention may include one or more photosensitizers. Nanoparticles include, for example, nanocapsules and nanovectors.

By “subject” is meant a mammal, such as a human patient or an animal (e.g., a rodent, bovine, equine, porcine, ovine, canine, feline, or other domestic mammal).

A “therapeutically effective amount” is an amount sufficient to effect a beneficial or desired clinical result.

By “treat” is meant stabilize, reduce, or ameliorate the symptoms of any disease or disorder.

By “undesired cell” is meant a cell whose elimination or modulation is useful for the treatment of a disease or disorder (e.g., age-related macular degeneration, an immunoinflammatory disorder, rheumatoid arthritis, a neoplasm, or a pathogen infection).

### METHODS OF THE INVENTION

The invention features compositions and methods useful for the elimination of an undesired cell or modulation of an undesired cell. Accordingly, compositions and methods are provided for the treatment of age-related macular degeneration, rheumatoid arthritis, a neoplasm or a pathogen infection in a subject using nanoparticles of the invention, where the nanoparticles contain a photosensitizer core, a polymer shell, and a targeting aptamer fixed to the surface of the shell.

### Photosensitizer Compositions

Photosensitizers known in the art are typically selected for use according to: 1) efficacy in delivery, 2) proper localization in target tissues, 3) wavelengths of absorbance, 4) proper excitatory wavelength, 5) purity, and 6) in vivo effects on pharmacolinetics, metabolism, and reduced toxicity.

Photosensitizers are chemical compounds that produce a biological effect upon photoactivation or a biological precursor of a compound that produces a biological effect upon photoactivation. Typically, photoactivation of the photosensitizer results in cytotoxicity. Photosensitizers of the invention can be any Inown in the art, including photofrin.RTM, synthetic diporphyrins and dichlorins, phthalocyanines with or without metal substituents, chloroaluminum phthalocyanine with or without varying substituents, O-substituted tetraphenyl porphyrins, 3,1-meso tetrakis (o-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AIPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

In a preferred embodiment, the photosensitizer is a benzoporphyrin derivative (“BPD”), such as BPD-MA, also commercially known as BPD Verteporfin (“BPD”). In another embodiment, the photosensitizer is a phenothiazine or a phenoloxazine. U.S. Pat. No. 4,883,790 describes BPDs. BPD is a so-called second-generation compound which lacks the prolonged cutaneous phototoxicity of Photofrin®. BPD has been thoroughly characterized, and it has been found to be a highly potent photosensitizer for PDT.

In specific embodiments, photosensitizers of the present invention absorb light at a relatively long wavelength, thereby absorbing at low energy. Low-energy light can travel further through tissue than high-energy light, which becomes scattered. Optimal tissue penetration by light occurs between about 650 and about 800 nm. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 mn) or bacteriochlorins found in photosynthetic bacteria (about 750 to about 820 nm).

Photosensitizers of the invention can be any known in the art, and optionally coupled to molecular carriers.
i) Porphyrins and Hydroporphyrins

Porphyrins and hydroporphyrins can include, but are not limited to, Photofrin® RTM (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methyl pyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(III) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generation porphyrins.

Chlorins and bacteriochlorins are also porphyrin derivatives, however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to about 740 nm. A specific embodiment of the invention uses chlorinₑ₆.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. In a specific embodiment, the photosensitizer can be Antrin®, otherwise known as motexafin lutetium.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin Ix is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is Mv0633 (Miravant). MV0633 is well suited for cardiovascular therapies and as such, can be used in therapeutic and diagnostic methods of the invention. In another specific embodiment, the photosensitizer is a phenothiazine or a phenoloxazine.

In specific embodiments, the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength. Such specific embodiments include motexafin lutetium (Antrin®) and chlorinₑ₆.
ii) Cyanine and other Photoactive Dyes

Cyanine and other dyes include but are not limited to merocyanines, phthalocyanines with or without metal substituents, chloroaluminum phthalocyanine with or without varying substituents, sulfonated aluminum PC, ring-substituted cationic PC, sulfonated AlPc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds and phenothiazine derivatives including methylene blue. Cyanines are deep blue or purple compounds that are similar in structure to porphyrins.

However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.
iii) Other Photosensitizers

Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e. mitoxantrone, doxorubicin). Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics such as tetracyclines and fluoroquinolone compounds have demonstrated photoactive properties.
iv) Devices and Methods For Photoactivation

Typically, administration of photosensitizers is followed by a sufficient period of time to allow accumulation of the photosensitizer at the target site. Following this period of time, the photosensitizer is activated by irradiation. This is accomplished by applying light of a suitable wavelength and intensity, for an effective length of time, at the site of the inflammation. As used herein, “irradiation” refers to the use of light to induced a chemical reaction of a photosensitizer.

The suitable wavelength, or range of wavelengths, will depend on the particular photosensitizer(s) used, and can range from about 450 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, photosensitizers with strong absorbances in the range of about 600 nm to about 900 nm are optimal for PDT. For photoactivation, the wavelength of light is matched to the electronic absorption spectrum of the photosensitizer so that the photosensitizer absorbs photons and the desired photochemistry can occur. Wavelength specificity for photoactivation generally depends on the molecular structure of the photosensitizer. Photoactivation can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photosensitizer can alter these values. In general, photosensitizers with longer absorbing wavelengths and higher molar absorption coefficients at these wavelengths are more effective photodynamic agents.

Photoactivating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art.

The light for photoactivation can be produced and delivered to the site of inflammation by any suitable means known in the art. Photoactivating light can be delivered to the site of inflammation from a light source, such as a laser or optical fiber. Preferably, optical fiber devices that directly illuminate the site of inflammation deliver the photoactivating light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of photosensitizer composition, and light activating the photosensitizer composition, is administered in an amount sufficient to produce a phototoxic species. For example, where the photosensitizer is chlorinₑ₆ administration to humans is in a dosage range of about 0.5 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 20-500 J/cm, preferably about 50 to about 300 J/cm and more preferably about 100 to about 200 J/cm.

The fluence rate is in the range of about 20 to about 500 mw/cm, preferably about 50 to about 300 mw/cm and more preferably about 100 to about 200 mw/cm. There is a reciprocal relationship between photosensitizer compositions and light dose, thus, determination of suitable wavelength, light intensity, and duration of illumination is within ordinary skill in the art.

The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorinₑ₆, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

### Nanoparticles

A nanoparticle of the invention may include one or more photosensitizers. In addition, each nanoparticle may include on its surface one or more aptamers. Nanoparticles of the invention are typically about 30-60 KD. In one embodiment, the size of nanoparticles of the invention is between about 100-500 nm. Preferably, the size of the nanoparticles of the invention is about 200-300 nm or less. Methods of producing nanoparticles are known in the art and are described herein. Exemplary methods for producing nanoparticles or nanocapsules of the invention are described, for example, in U.S. Pat. Nos. 4,452,973, 4,716,203, 4,862,168, 4,942,035, 5,714,166, 5,683,723, 5,384,333, 5,407,609, 5,449,513, 5,476,909, 5,510,103, 5,543,158, 5,548,035, 5,702,717, and 5,919,442, 6,312,731, 6,569,463, 6,599,519, 6,602,932, 6,689,468, 6,713,533, and 6,716,450. Desirably, the nanoparticles are of small size (<100 nm) and narrow size dispersity; and have the ability to localize biomolecules in the shell, or capsule, interior.

Targeted nanoparticles are typically multicomponent structures with a carrier system that forms the core and contains the therapeutic or imaging payload, surface modifiers to reduce reticuloendothelial system uptake and enhance biodistribution, and a targeting component. Treating solid tumors with target-specific drugs has received intense attention for many years. In order for macromolecule drugs to reach cancer cells within solid tumors, two major barriers have to be overcome: the administered macromolecules have to survive degradation, and pass through the reticuloendothelial system (RES), which is designed to sequester macromolecules, both relatively small (<70 nm) and large (>300 nm), from circulation.

Nanoparticles are a colloidal carrier system that has been shown to improve the efficacy of the encapsulated drug by prolonging the serum half-life and reducing the uptake by RES. Polyalkylcyanoacrylates (PACAs) nanoparticles are a polymer colloidal drug delivery system that is in clinical development for cancer therapy as described by Stella et al., J. Pharm. Sci., 2000. 89: p. 1452-1464; Brigger et al., Int. J. Pharm., 2001. 214: p. 37-42; Calvo et al., Pharm. Res., 2001. 18: p. 1157-1166; and Li et al., Biol. Pharm. Bull., 2001. 24: p. 662-665. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence in the bloodstream.

Nanoparticles are synthesized using virtually any biodegradable shell known in the art. In one embodiment, a polymer, such as poly (lactic-acid) (PLA) or poly (lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation (Gref et al., Science 263(5153): 1600-1603, 1994). Alternatively, the COOH group is converted to an N-hydroxysuccinimide (NHS) ester for covalent conjugation to amine-modified aptamers (Farolhzad et al., Cancer Res,. 64: 7668-7672, 2004).

Biocompatible polymers useful in the composition and methods of the invention include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulphate sodium salt, poly(methyl methacrylate), poly(ethylmethacrylate), poly(butylmethacrylate), poly(isobutylmethacryla-te), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the invention include PEG-PLGA polymers.

In response to the growing need for encapsulation materials, several different routes to producing hollow polymeric capsules are available. In one example, the shell is composed of dendrimers (Zhao, M., et al. J. Am. Chem. Soc. (1998) 120:4877). A dendrimer is an artificially manufactured or synthesized large molecule comprised of many smaller ones linked together—built up from branched units called monomers. Technically, dendrimers are a unique class of a polymer, about the size of an average protein, with a compact, tree-like molecular structure, which provides a high degree of surface functionality and versatility. Their shape gives them vast amounts of surface area, making them useful building blocks and carrier molecules at the nanoscale and they come in a variety of forms, with different physical (including optical, electrical and chemical) properties. In other embodiments, the shell comprises block copolymers (Thurmond, K. B., II, et al. J. Am. Chem. Soc. (1997) 119:6656; Macknight, W. J., et al., Acc. Chem. Res. (1998) 31:781; Harada, A. and Kataoka, K. Science (1999), 283:65), vesicles (Hotz, J. and Meier, W. Langmuir (1998) 14:1031; Discher, B. M., et al., Science (1999) 284:1143), hydrogels (Kataoka, K. et al. J. Am. Chem. Soc. (1998) 120:12694) and template-synthesized microtubules (Martin, C. R. and Parthasarathy, R. V. Adv. Mater. (1995) 7:487) that are capable of encapsuling a photosensitizer.

### Aptamers

Nucleic acid aptamers are single-stranded nucleic acid (DNA or RNA) ligands that function by folding into a specific globular structure that dictates binding to target proteins or other molecules with high affinity and specificity, as described by Osborne et al., Curr. Opin. Chem. Biol. 1:5-9, 1997; and Cerchia et al., FEBS Letters 528:12-16, 2002. Desirably, the aptamers are small, approximately ˜15KD. The aptamers are isolated from libraries consisting of some 10¹⁴-10¹⁵ random oligonucleotide sequences by a procedure termed SELEX (systematic evolution of ligands by exponential enrichment). See Tuerk et al., Science, 249:505-510, 1990; Green et al., Methods Enzymology. 75-86, 1991;Gold et al., Annu. Rev. Biochem., 64: 763-797, 1995; Uphoff et al., Curr. Opin. Struct. Biol., 6: 281-288, 1996. Methods of generating aptamers are known in the art and are described, for example, in U.S. Pat. Nos. 6,344,318, 6,331,398, 6,110,900, 5,817,785, 5,756,291, 5,696,249, 5,670,637, 5,637,461, 5,595,877, 5,527,894, 5,496,938, 5,475,096, 5,270,163, and in U.S. Patent Application Publication Nos. 20040241731, 20030198989, 20030157487, and 20020172962.

Methods for affixing an aptamer to a nanoparticles are known in the art. See, for example, U.S. Patent Publication Nos. 20060014172, 20050250094, 20030143598. In fact, any suitable method for attaching a nucleic acid molecule to a solid substrate may be used. In one embodiment, a salt aging method for preparing nanoparticle-oligonucleotide conjugates is used, as described in U.S. Pat. No. 6,506,564, in PCT/US97/12783, PCT/US00/17507, PCT/US01/01190, and PCT/US01/10071, each of which is incorporated by reference in its entirety. Aptamers and oligonucleotides having covalently bound thereto a moiety comprising a functional group which can bind to the nanoparticles are used. The moieties and functional groups are those described in U.S. Pat. Nos. 6,506,564 and 6,767,702 (which are incorporated by reference in its entirety) for binding (i.e., by chemisorption or covalent bonding) oligonucleotides to nanoparticles. For instance, oligonucleotides having an alkanethiol or an alkanedisulfide covalently bound to their 5′ or 3′ ends can be used to bind the oligonucleotides to a variety of nanoparticles. Thioaptamers having phosphorothioate or phosphorodithioate functional moieties covalently bound to their 5′ or 3′ ends can be used to bind the aptamers to a variety of nanoparticles. Additionally, the oligonucleotides can be bound through an oligonucleotide tail such as a polyA tail which has a high affinity for the nanoparticle surface (see Tarlov and coworkers, JACS, 2004). Alternatively, streptavidin or x-biotin modified nanoparticles can be contacted with biotinylated aptamers to form the aptamer nanoparticle conjugate.

The aptamers are contacted with the nanoparticles in water for a time sufficient to allow at least some of the aptamers and oligonucleotides to bind to the nanoparticles by means of the functional groups. Such times can be determined empirically. For instance, it has been found that a time of about 12-24 hours gives good results. Other suitable conditions for binding of the aptamers and oligonucleotides can also be determined empirically.

Next, at least one salt is added to the water to form a salt solution. The salt can be any water-soluble salt. For instance, the salt may be sodium chloride, magnesium chloride, potassium chloride, ammonium chloride, sodium acetate, ammonium acetate, a combination of two or more of these salts, or one of these salts in phosphate buffer. Preferably, the salt is added as a concentrated solution, but it could be added as a solid. The salt can be added to the water all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the oligonucleotides from each other and, either the electrostatic attraction of the negatively-charged oligonucleotides for positively-charged nanoparticles, or the electrostatic repulsion of the negatively-charged oligonucleotides from negatively-charged nanoparticles. Gradually reducing the electrostatic attraction and repulsion by adding the salt gradually over time has been found to give the highest surface density of oligonucleotides on the nanoparticles. Suitable ionic strengths can be determined empirically for each salt or combination of salts. A final concentration of sodium chloride of from about 0.1 M to about 1.0 M in phosphate buffer, preferably with the concentration of sodium chloride being increased gradually over time, has been found to give good results.

After adding the salt, the aptamers, oligonucleotides and nanoparticles are incubated in the salt solution for an additional period of time sufficient to allow sufficient additional oligonucleotides to bind to the nanoparticles to produce the stable nanoparticle conjugates having aptamers and oligonucleotides bound thereto. As will be described in detail below, an increased surface density of the oligonucleotides on the nanoparticles has been found to stabilize the conjugates. The time of this incubation can be determined empirically. In one embodiment, a total incubation time of about 24-48 is used. The salt concentration can be increased gradually over this time. A surface density adequate to make the nanoparticles stable and the conditions necessary to obtain it for a desired combination of nanoparticles and aptamers/oligonucleotides can be determined empirically.

The aptamer fixed to a nanoparticle is capable of binding with specificity to a marker present on the surface of a neoplastic cell. “Binding with specificity” means that noncancer cells are either not specifically bound by the aptamer or are only poorly bound by the aptamer. In general, aptamers typically have binding constants in the picomolar range. Particularly useful in the methods of the invention are aptamers having apparent dissociation constants of about 1, 10, 15, 25, 50, 75, or 100 nM. Because many neoplasms contain a heterogenous population of cells, in one embodiment, a nanoparticle of the invention contains two or more aptamers, each of which recognizes a different protein. In some embodiments, a nanoparticle composition comprises at least two nanoparticles, where each nanoparticle is targeted to a different protein.

In one embodiment, ErbB3 is the molecular target of the nanoparticle. The sequence of ErbB3-specific aptamer has been published by Chen et al., (PNAS,. 100(16): 9226-9231, 2003), who showed that high-affinity binding of this aptamer inhibited hrg-dependent tyrosine phosphorylation of HER2 and the hrg-induced growth response of MCF7 cells. Because aptamers can act as direct antagonists of the biological function of proteins, PhotoSensitizer Nanoparticle Aptamer Conjugate Therapy (SNACT) may be viewed as a dual therapy. The therapeutic benefit of SNACT derives both from the biological antagonism caused by aptamer binding and from the cytotoxicity upon light activation of the photosensitizer.

The invention encompasses stabilized aptamers having modifications that protect against 3′ and 5′ exonucleases as well as endonucleases. Such modifications desirably maintain target affmity while increasing aptamer stability in vivo. In various embodiments, aptamers of the invention include chemical substitutions at the ribose and/or phosphate and/or base positions of a given RNA sequence. For example, aptamers of the invention include chemical modifications at the 2′ position of the ribose moiety, circularization of the aptamer, 3′ capping and ‘spiegelmer’ technology. Such modifications are known in the art and are described for example in Reference Nos: 4-9. Aptamers having A and G nucleotides sequentially replaced with their 2′-OCH3 modified counterparts are particularly useful in the methods of the invention. Such modifications are typically well tolerated in terms of retaining aptamer affinity and specificity (Burmeister et al., Chemistry & Biology, 12(1): 25-33, 2005). In various embodiments, aptamers include at least about 10%, 25%, 50%, or 75% modified nucleotides. In other embodiments, as many as about 80-90% of the aptatmer's nucleotides contain stabilizing substitutions. In other embodiments, 2′-OMe aptamers are synthesized. Such aptamers are desirable because they are inexpensive to synthesize and natural polymerases do not accept 2′-OMe nucleotide triphosphates as substrates so that 2′-OMe nucleotides cannot be recycled into host DNA. A fully 2′-O-methyl aptamer, named ARC245, was reported to be so stable that degradation could not be detected after 96 hours in plasma at 37° C. or after autoclaving at 125° C. (Burmeister et al., Chemistry & Biology, 12(1): 25-33, 2005). Using methods described herein, aptamers will be selected for reduced size and increased stability. In one embodiment, aptamers having 2′-F and 2′-OCH₃ modifications are used to generate miniaturized nuclease resistant ovarian cancer-specific nanoparticles. Other modifications that stabilize aptamers are known in the art and are described, for example, in U.S. Pat. Nos. 5,580,737; and in U.S. Patent Application Publication Nos. 20050037394, 20040253679, 20040197804, and 20040180360.

Using standard methods tumor-specific aptamers can be selected that bind virtually any tumor marker known in the art. Markers to which tumor-specific aptamers bind are also well known in the art. For example, markers bound by the tumor-specific aptamers of the invention include, but are not limited to, those known in the art to be present on CA-125 (e.g., Genbank Accession No. NP_{—}078966), gangliosides G(D2), G(M2) and G(D3), CD20 (e.g., Genbank Accession No. P11836), CD52 (e.g., Genbank Accession No. NP_{—}001794), CD33 (e.g., Genbank Accession No. NP_{—}001763), Ep-CAM (e.g., Genbank Accession No. P16422), CEA (e.g., Genbank Accession No. AAA51972), bombesin4ike peptides (e.g., NP_{—}002082, NP_{—}001012530), prostate specific antigen (PSA) (e.g., Genbank Accession No. CAD30844, CAD54617, CAD30845), prostate-specific membrane antigen (PSMA) (e.g., Genbank Accession No. AAC83972), HER2/neu (e.g., Genbank Accession No. AAD56009), epidermal growth factor receptor (e.g., Genbank Accession No. 1006266A), erbB2 (e.g., Genbank Accession No. AAD56009), erbB3 (e.g., Genbank Accession No. P21860), erbB4 (e.g., Genbank Accession No. DAA00042, NP_{—}039250, ), CD44v6 (e.g., Genbank Accession No. AAB13626, AAB 13622, AAB13623), Ki-67 (e.g., Genbank Accession No. CAA46519, CAA46520), VEGF (e.g., Genbank Accession No. AAA35789), VEGFRs (e.g., Genbank Accession No. CAA61916), VEGFR3 (e.g., Genbank Accession No. AA089505), estrogen receptors (e.g., Genbank Accession No. P03372), Lewis-Y antigen, TGFβ1 (e.g., Genbank Accession No. AAX59023), IGF-1 receptor (e.g., Genbank Accession No. NP_{—}000866), EGF (e.g., Genbank Accession No. NP_{—}001954), EGFα, c-Kit receptor (e.g., Genbank Accession No. NP_{—}002825, AAB21235), transferrin receptor (e.g., Genbank Accession No. NP_{—}003225), IL-2R (e.g., Genbank Accession No. NP_{—}000869), CO17-1A (Oredipe et al., Hybridoma. 11(5):607-15, 1992), tumor-associated antigen MUCI (e.g., Genbank Accession No. NP_{—}001018017), TGF beta receptor (e.g., Genbank Accession No. NP_{—}004603, NP_{—}003234), and TGF beta (e.g., Genbank Accession No. NP_{—}000651, NP_{—}003230). Aptamers of the invention can recognize tumors derived from a wide variety of tissue types, including, but not limited to, breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

Exemplary aptamers useful for targeting a neoplastic cell include those that target human epidermal growth factor receptor-3 (HER3) (See Chen et al., Proc Natl Acad Sci U S A. 2003 Aug. 5; 100(16):9226-31), human receptor tyrosine kinases, such as RET (See Cerchia et al., PLoS Biol. 2005 April; 3(4): el23), human serine/threonine kinases, such as Raf-l (See Kimoto et al., Eur. J. Biochem. 269, 697-704 (2002); VEGF165 (See Green et al., J Biol Chem. 1998 Aug. 7; 273(32):20556-67); PSMA (Lupold et al., Cancer Research 62: 4029-4033), and platelet-derived growth factor receptor (See Pietras et al., Cancer Res. 2001 Apr. 1; 61(7):2929-34; Floege et al., Am J Pathol. 1999 January; 154(1):169-79). Other aptamers useful in the methods of the invention are publically available at the Aptamer Database (University of Texas at Austin, Inst. for Cellular and Mol. Bio) which is available through the on-line web site at http:/aptamer.icmb.utexas.edu.

Exemplary aptamers useful for targeting an angiogenic cell type present, for example, in tumors, include EYE0001, and those that target angiopoietin-2 (White et al., Proc Natl Acad Sci U S A. 2003 Apr. 29; 100(9):5028-33 and pigpen (Blank et al., J Biol Chem. 2001 May 11; 276(19):16464-8).

### Therapy

Compositions and methods of the invention are useful for the elimination or modulation of undesired cells. In one example, the undesired cells are neoplastic cells. Examples of neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

In one example, the present invention provides compositions and methods for reducing growth of a tumor cell in a subject, wherein tumoricidal aptamers bind with specificity to a cell surface epitope (or epitope of a receptor-binding molecule) of a neoplastic cell or a cell that is involved in the growth and/or propagation of a neoplastic cell such as a cell comprising the vasculature of a tumor or blood vessels that supply tumors and/or stromal cells.

A tumor comprises one or more neoplastic cells, or a mass of neoplastic cells, and can also encompass cells that support the growth and/or propagation of a neoplastic cell, such as vasculature and/or stroma, but not necessarily macrophages. In one example, the present invention provides compositions and methods for reducing or stabilizing the growth and/or proliferation of a tumor cell in a subject, wherein a nanoparticle of the invention binds with specificity to cell surface epitopes (or epitopes of receptor-binding molecules) of a neoplastic cell or a cell that is involved in the growth and/or propagation of a neoplastic cell such as a cell within the vasculature of a tumor or blood vessels that supply tumors and/or stromal cells.

The lymphatic system is the primary pathway for the metastasis of most cancers. Activation of lymphatic endothelium by lymphangiogenic factors directly influences tumor progression by promoting tumor cell invasion and migration into the lymphatic vessels. VEGF-C and VEGF-D are members of the vascular endothelial growth factor (VEGF) family of angiogenic growth factors that have been identified as growth factors for lymphatic vessels. The induction of tumor lymphangiogenesis by VEGF-C results in increased infiltration of lymphatic vessels by tumor cells, and the extent of intratumoral lymphangiogenesis directly relates to the extent of tumor metastases. VEGFR-3, the receptor for VEGF-C and VEGF-D, is expressed in all tumor-associated lymphatic vessels and has been implicated in tumor lymphangiogenesis.

Methods of this invention are particularly suitable for administration to humans with neoplastic diseases. Especially relevant are melanoma, neuroblastoma, glioma, sarcoma, lymphoma, ovarian, prostate, colorectal and small cell lung cancers. The methods comprise administering an amount of a pharmaceutical composition containing a nanoparticle of the invention, where the nanoparticle includes a photosensitizer core, a polymer shell, and a targeting aptamer fixed to the surface, effective to achieve the desired effect, be it palliation of an existing tumor mass or prevention of recurrence.

Methods of the invention are particularly suitable for use in the primary treatment of intraperitoneal neoplasms, such as ovarian and colorectal cancers and cancer of the bladder. In one embodiment, the aptamer specifically targets a cell surface molecule expressed on an intraperitoneal neoplasm. In one embodiment, the methods of the invention are used in combination with any conventional therapy or therapies. Currently, advanced ovarian cancer is treated by staging/debulking surgery, followed by chemotherapy. Typically chemotherapy for ovarian cancer involves treatment with a combination of Taxol and a platinum-based regimen. Rather than chemotherapy, combination therapy may be administered. For example, an administration scheme is envisioned whereby a nanoparticle composition is administered either before or after maximal debulking. Following the surgical procedure light activation is administered in order to eliminate residual cancer cells.

Methods of the invention are particularly suitable for use in treating and imaging brain cancer. When the site of delivery is the brain, the therapeutic agent is advantageously delivered to the brain. The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may involve the use of specific drug delivery strategies. Delivery of therapeutic agents to the CNS can be achieved by several methods.

One method relies on neurosurgical techniques. In the case of gravely ill patients, surgical intervention is warranted despite its attendant risks. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes (Neuwelt and Rapoport, Neurosurgery 14(2):154-60, 1984), (Baba et al., J Cereb Blood Flow Metab. 11(4):638-43, 1991), (Genmuso et al., Cancer Invest., 11(2):118-28, 1993).

Further, it may be desirable to administer the compositions locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

Accordingly, an embodiment of the invention relates to a method of stabilizing, reducing, or inhibiting neoplastic cell growth and/or proliferation in a subject comprising the steps of administering a therapeutically effective amount of at least one nanoparticle composition to a neoplastic cell, wherein the aptamer on the nanoparticle binds with specificity to a cell surface marker of the neoplastic cell; localizing the nanoparticle to the neoplastic cell; light activating the neoplastic cell to produce reactive species; and stabilizing, reducing, or inhibiting the growth and/or proliferation of the neoplastic cell.

Desirably, compositions of the invention induce cytotoxicity in any undesired cell. Assays for measuring cytotoxicity are known in the art, and are described, for example, by Crouch et al. (J. Inmunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1:611, 1991; Cory et al., Cancer Comm. 3, 207-12, 1991; Paull J. Heterocyclic Chem. 25, 911, 1988). Assays for cell viability are also available commercially. These assays include CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay

In one embodiment, nanoparticles of the invention induce the death of an undesired cell via apoptosis or necrosis. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

In other embodiments, the undesired cell functions in a disease or disorder, where elimination or modulation of the cell ameliorates, stabilizes, or treats the disease or disorder. Exemplary diseases susceptible to treatment using a method of the invention are age-related macular degeneration, rheumatoid arthritis, immunoinflammatory disorders, and hyperproliferative disorders.

Accordingly, an embodiment of the invention relates to a method of stabilizing, reducing, or inhibiting a disease or disorder in a subject in need thereof comprising the steps of contacting an undesired cell with a therapeutically effective amount of a nanoparticle of the invention, wherein the aptarner on the nanoparticle binds with specificity to a cell surface marker of the undesired cell; localizing the nanoparticle to the undesired cell; light activating the undesired cell to produce a rective species; and stabilizing, reducing, or inhibiting the disease or disorder by eliminating or modulating the undesired cell.

In one embodiment, a nanoparticle of the invention comprises an EYE0001 aptamer affixed to its polymer shell. In another embodiment, arthritis is treated using a nanoparticle that includes an aptamer that targets an arthritis disease marker, such as an Oncostatin M receptor, TNF receptor, or CD134, a marker that is expressed on auto-aggressive T cells. In another embodiment, the undesired cell is a a cell of a pathogen or a cell infected with a pathogen. Pathogens include, but are not limited to, bacteria, viruses, fingi, and parasites. Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, Corynebacterium diphtheriae, Corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Legionella, Leptospira, Listeria, Morganella, Moraxella, Mycobacterium, Neisseria, Pasteurella, Pasturella multocida, Proteus, Providencia, Pseudoinonas, Rickettsia, Salmonella, Serratia, Shigella, Staphylococcus, Stentorophonmonas, Streptococcus, Streptobacillus moniliformis, Treponema, Treponema pallidium, Treponena pertenue, Xanthomonas, Vibrio, and Yersinia.

Both gram negative and gram positive bacteria may act as pathogens in vertebrate animals. Gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacterpyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumioniae, pathogenic Campylobacter sp., Enterococcus sp., Haemophilus influenzae, Bacillus antracis, corynebacterium diphtheriae, corynebacterium sp., Erysipelothrix rhusiopathiae, Clostridiun perfringers, Clostridiunz tetani, Enterobacter aerogenes, Klebsiella pneumoniae, Pasturella multocida, Bacteroides sp., Fusobacterium nucleatum, Streptobacillus moniliforinis, Treponena pallidium, Treponema pertenue, Leptospira, Rickettsia, and Actinonzyces israelli.

Examples of viruses that have been found in humans include, but are not limited to, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of pathogenic fungi include, without limitation, Alternaria, Aspergillus, Basidiobolus, Bipolaris, Blastomyces dermatitidis, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, Candida lusitaniae, Coccidioides, Coccidioides immitis, Cladophialophora, Chlamydia trachomatis, Candida albicans, Cryptococcus, Cryptococcus neoformans, Cunninghamella, Curvularia, Exophiala, Fonsecaea, Histoplasma, Histoplasma capsulatum, Madurella, Malassezia, Plastoinyces, Rhodotorula, Scedosporium, Scopulariopsis, Sporobolomyces, Tinea, and Trichosporon.

Examples of parasites include Acanthamoeba, Babesia, Babesia microti, Babesia divergens, Cryptosporidium, Eimeria, Entamoeba histolytica, Enterocytozoon bieneusi Giardia lamblia, Isospora, Leishmania, Leishmania tropica, Leishmania braziliensis, Leishmania donovani, Naegleria, Neospora, Plasmodium, Sarcocystis, and Schistosoma Trypanosoma ciuzi, Toxoplasma gondii, and Trichinella spiralis. Exemplary parasitic helminths include nematodes, cestodes, and trematodes. Preferred nematodes include filariid, ascarid, capillarid, strongylid, strongyloides, trichostrongyle, and trichurid nematodes.

Other medically relevant microorganisms have been described extensively in the literature, e.g., see C. G. A Thomas, Medical Microbiology, Bailliere Tindall, Great Britain 1983, the entire contents of which is hereby incorporated by reference.

Accordingly, an embodiment of the invention relates to a method of stabilizing, reducing, or ameliorating a pathogen infection in a subject comprising the steps of: contacting a pathogen or a cell infected with a pathogen with a therapeutically effective amount of a nanoparticle of the invention, wherein the aptamer on the nanoparticle binds with specificity to a cell surface marker of the pathogen or infected cell; localizing the nanoparticle to the pathogen or infected cell; light activating the pathogen or infected cell to produce a reactive species (preferably at the site of the infection); and stabilizing, reducing, or ameliorating the pathogen infection.

Using standard methods pathogen-specific aptamers can be selected that bind virtually any pathogen marker known in the art. For example, pathogen-specific aptamers useful in the invention include, but are not limited to, those that bind a hepatitis protein, such as hepatitis C virus polymerase (Vo et al., Virology. 2003 Mar. 15; 307(2):301-16), hepatitis C virus nonstructural protein 3 protease (Fukuda et al., Eur J Biochem. 2000 June; 267(12):3685-94; Urvil et al., Eur J Biochem. 1997 Aug. 15; 248(1):130-8), hepatitis B virus core protein (Butz et al., Oncogene. 2001 Oct. 4; 20(45):6579-86), and HCV NS3 helicase domain (Nishikawa et al., Oligonucleotides. 2004; 14(2): 114-29); a human immunodeficiency virus protein, such as HIV-1 reverse transcriptase (Nickens et al., RNA. 2003 September; 9(9):1029-33), HIV-1 gp120 (glycoprotein) (Khati et al., J. Virol. (2003), Vol. 77, Issue 23, pages 12692-8); and human immunodeficiency virus type-1 nucleocapsid protein (Kim et al., Biochem Biophys Res Commun. 2002 Mar. 8; 291(4):925-31); an Epstein-Barr virus protein, such as Epstein-Barr ribosomal L22 protein (Dobblestein et al., J. Virol. (1995), Vol. 69, Issue 12, pages 8072-34); or an influenze viral protein, such as viral hemagglutinin (Jeon et al., J Biol Chem. 2004 Nov. 12; 279(46):48410-9).

Also useful in the methods of the invention are those aptamers that bind a bacterial protein. Such aptamers include, but are not limited to those that bind Bacillus anthracis spores (Bruno et al., Biosens Bioelectron. 1999 May 31; 14(5):457-64; Zhen et al., Sheng Wu Hua Xue Yu Sheng Wu Wu Li Xue Bao (Shanghai). 2002 September; 34(5):635-42); Escherichia coli SelB protein (Kug et al., RNA. 1999 September; 5(9): 1180-90); MS2 coat protein (Hirao et al., Mol Divers. 1998-99; 4(2):75-89); Yersiniapestis YopM (Skrzypek et al., Thromb Res. 1996 Oct. 1; 84(1):33-43).

Other aptamers that are useful in the methods of the invention include, but are not limited to those that bind a parasitic protein from Trypanosoma cruzi (Ulrich et al., Biol Chem. 2002 Jun. 7; 277(23):20756-62); or an African trypanosome protein (Homann et al., Bioorg Med Chem. 2001 October; 9(10):2571-80)

### Photoactivation

Photoactivating light can be delivered to an undesired cell, a tumor site or to a pathogen using a conventional light source or from a laser. While nanoparticles of the invention desirably include a biodegradable polymer, degradation of this polymer is not required for photoactivation of the photosensitizer. Target tissues are illuminated, usually with red light from a laser. Given that red and/or near infrared light best penetrates mammalian tissues, photosensitizers with strong absorbances in the approximately 600 nm to 900 nm range are optimal for PDT. Delivery can be direct, by transillumination, or by optical fiber.

Optical fibers can be connected to flexible devices such as balloons equipped with light scattering medium. Flexible devices can include, for example, laproscopes, arthroscopes and endoscopes.

Following administration of the nanoparticle composition, it is necessary to wait for the photosensitizer to reach an effective tissue concentration at the neoplasm site before photoactivation. Duration of the waiting step varies, depending on factors such as route of administration, tumor location, and speed of photosensitizer movement in the body. In addition, where a nanoparticle composition targets receptors or receptor binding epitopes, the rate of nanoparticle composition uptake can vary, depending on the level of receptor expression and/or receptor turnover on undesired cells. For example, where there is a high level of receptor expression, the rate of nanoparticle composition binding and uptake is increased. The waiting period should also take into account the rate at which the nanoparticle is degraded and the photosensitizer within is dequenched in the target tissue. Determining a useful range of waiting step duration is within the ordinary skill in the art and may be optimized by utilizing fluorescence optical imaging techniques.

Following the waiting step, the nanoparticle composition is activated by photoactivating light applied to the undesired cell, tumor site, or pathogen. This is accomplished by applying light of a suitable wavelength and intensity, for an effective length of time, specifically to the lesion site. The suitable wavelength, or range of wavelengths, will depend on the particular photosensitizer(s) used. Wavelength specificity for photoactivation depends on the molecular structure of the photosensitizer. Photoactivation occurs with sub-ablative light doses. Determination of suitable wavelength, light intensity, and duration of illumination is within ordinary skill in the art.

The light for photoactivation can be produced and delivered to the undesired cell, tumor site, or pathogen by any suitable means. For superficial undesired cells, tumor sites, pathogens, or open surgical sites, suitable light sources include broadband conventional light sources, broad arrays of light emitting diodes (LED), and defocussed laser beams.

For non-superficial lesion sites, including those in intracavitary settings, the photoactivating light can be delivered by optical fiber devices. For example, the light can be delivered by optical fibers threaded through small gauge hypodermic needles. Optical fibers also can be passed through arthroscopes, endoscopes and laproscopes. In addition, light can be transmitted by percutaneous instrumentation using optical fibers or cannulated waveguides.

Photoactivation at non-superficial lesion sites also can be by transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, i.e. lightboxes or convergent light beams.

For photoactivation, the wavelength of light is matched to the electronic absorption spectrum of the photosensitizer so that photons are absorbed by the photosensitizer and the desired photochemistry can occur. Except in special situations, where the undesired cells being treated are very superficial, the range of activating light is typically between approximately 600 and 900 nm. This is because endogenous molecules, in particular hemoglobin, strongly absorb light below about 600 nm and therefore capture most of the incoming photons (Parrish, 1978). The net effect would be the impairment of penetration of the activating light through the tissue. The reason for the 900 nm upper limit is that energetics at this wavelength may not be sufficient to produce ¹O₂, the activated state of oxygen, which without wishing to necessarily be bound by any one theory, is perhaps critical for successful PDT. In addition, water begins to absorb at wavelengths greater than about 900 nm. While spatial control of illumination provides specificity of tissue destruction, it can also be a limitation of PDT. Photoactivation is feasible when target sites are accessible to light delivery systems. Accordingly, issues of light dosimetry need to be addressed (Wilson, 1989). In general, the amenability of lasers to fiberoptic coupling makes the task of light delivery to most anatomic sites manageable, although precise dosimetry remains complex and elusive.

The effective penetration depth, δeff, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (e.g., 700-800 nm) (Svaasand and Ellingsen, 1983). These values can be altered by altering the biologic interactions and physical characteristics of the photosensitizer. Factors such as self-shielding and photobleaching (self-destruction of the photosensitizer during the PDT) further complicate precise dosimetry. In general, photosensitizers with longer absorbing wavelengths and higher molar absorption coefficients at these wavelengths are more effective photodynamic agents.

### Nanoparticle Formulations

The compositions of the invention include a nanoparticle in a pharmaceutical excipient, where the nanoparticle contains a photosensitizer core, a polymer shell, and a targeting aptamer fixed to the surface of the polymer shell. Such nanoparticle compositions can be administered in any pharmaceutically acceptable excipient, such as water, saline, aqueous dextrose, glycerol, or ethanol. The compositions can also contain other medicinal agents, pharmaceutical agents, adjuvants, carriers, and auxiliary substances such as wetting or emulsifying agents, and pH buffering agents.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A therapeutically effective amount of a composition of the invention can be administered in one or more doses. An effective amount is an amount that is sufficient to palliate, ameliorate, reduce, stabilize, reverse or slow the progression of a disease or disorder, such as age-related macular degeneration, an inununoinflammatory disorder, such as rheumatoid arthritis, a hyperproliferative disease, or a neoplastic disease (e.g. tumors, dysplasias, leukemias) or otherwise reduce the pathological consequences of the neoplasm. A therapeutically effective amount can be provided in one or a series of administrations. In terms of an adjuvant, an effective amount is one sufficient to enhance the immune response to the immunogen. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the nanoparticle being administered.

The dosage of the nanoparticle (e.g., a nanoparticle containing a photosensitizer core, a polymer shell, and a targeting aptamer fixed to the surface) or nanoparticle composition can vary from about 0.01 mg/m² to about 500 mg/m², preferably about 0.1 mg/m² to about 200 mg/m², still more preferably about 0.1 mg/m² to about 10 mg/m². Ascertaining dosage ranges is well within the skill of one in the art. The dosage of nanoparticle (e.g., a nanoparticle containing a photosensitizer core, a polymer shell, and a targeting aptamer fixed to the surface) can range from about 0.1 to 10 mg/kg. Such dosages may vary, for example, depending on whether multiple administrations are given, tissue type and route of administration, the condition of the individual, the desired objective and other factors known to those of skill in the art. Administrations can be conducted infrequently, or on a regular weekly basis until a desired, measurable parameter is detected, such as diminution of disease symptoms. Administration can then be diminished, such as to a biweekly or monthly basis, as appropriate. Methods for administering photosensitizer compositions are known in the art, and are described, for example, in U.S. Pat. Nos. 5,952,329, 5,807,881, 5,798,349, 5,776,966, 5,789,433, 5,736,563, and 5,484,803.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with nanoparticle compositions. Therapeutic nanoparticle compositions (e.g., a nanoparticle containing a photosensitizer core, a polymer shell, and a targeting aptamer fixed to the surface of the shell in an appropriate excipient) are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

Another method of administration is intralesionally, for instance by direct injection directly into the tumor or undesired cell. Intralesional administration of various forms of immunotherapy to cancer patients does not cause the toxicity seen with systemic administration of immunologic agents (Fletcher and Goldstein, 1987), (Rabinowich et al., 1987), Rosenberg et al., 1986), (Pizza et al., 1984).

### Combination Therapy

Compositions and methods of the invention may be used in combination with any conventional therapy known in the art. In one embodiment, a nanoparticle composition of the invention that targets a neoplastic cell may be used in combination with any anti-neoplastic therapy known in the art. Exemplary anti-neoplastic therapies include, for example, chemotherapy, cryotherapy, hormone therapy, radiotherapy, and surgery. A nanoparticle composition of the invention may, if desired, include one or more chemotherapeutics typically used in the treatment of a neoplasm, such as abiraterone acetate, altretamine, anhydrovinblastine, auristatin, bexarotene, bicalutamide, BMS184476, 2,3,4,5,6-pentafluoro-N-(3-fluoro4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin- caleukoblastine, docetaxol, doxetaxel, cyclophosphamide, carboplatin, carmustine (BCNU),cisplatin, cryptophycin, cyclophosphamide, cytarabine, dacarbazine (DTIC), dactinomycin, daunorubicin, dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine. Other examples of chemotherapeutic agents can be found in Cancer Principles and Practice of Oncology by V. T. Devita and S. Hellman (editors), 6.sup.th edition (Feb. 15, 2001), Lippincott Williams & Wilkins Publishers.

In another embodiment, a nanoparticle composition of the invention that targets a pathogen cell may be used in combination with any anti-pathogen therapy known in the art. Exemplary anti-pathogen therapies include antibiotics, antivirals, fungicides, nematicides, and parasiticides, or any other biocide. Parasiticides are agents that kill parasites directly and can be used in combination with the methods and compositions described herein. Such compounds are known in the art and are generally commercially available. Exemplary parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivernectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Other anti-pathogen therapeutics useful in combination with a method of the invention include, but are not limited to, any one or more of the following: agent which reduces the activity of or kills a microorganism and includes but is not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloridc; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

### Kits or Pharmaceutical Systems

The present compositions may be assembled into kits or pharmaceutical systems for use in producing cytotoxic effects in undesired cells (e.g., neoplastic, proliferative, infected or pathogen cells). Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the agents of the invention.

The practice Qf the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

### Example 1

### Expression of ErbB3 Family Receptors in Ovarian Cancer

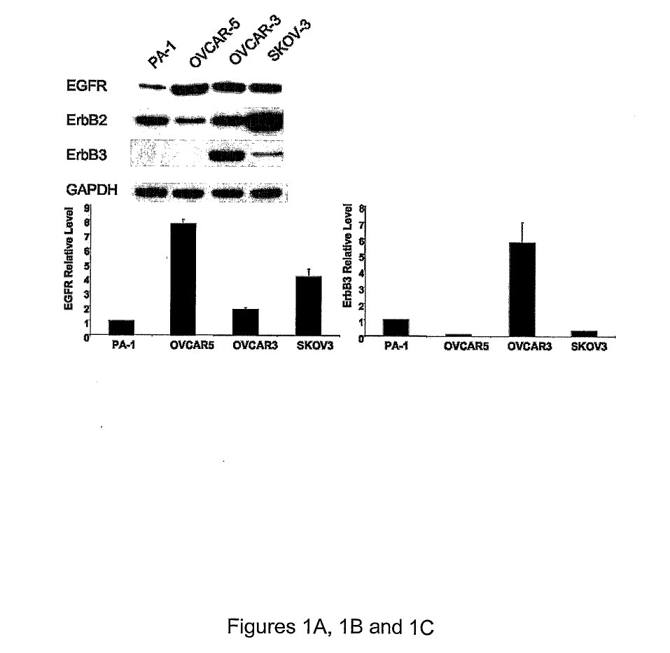

While the examples provided below relate to the use of the ErbB3 receptor, the skilled artisan appreciates that the invention is not so limited. The ErbB3 receptor is one exemplary target used to selectively target nanoparticles of the invention to a neoplastic cell. Virtually any marker expressed on the surface of an undesired cell, such as a neoplastic or pathogen cell, may be used to selectively target a nanoparticle of the invention to an undesired cell. FIG. 1A shows the expression levels of the ErbB family of receptors in various OVCA cell lines as determined by Western Blot. Total lysate (30 μg in 20 ul per lane) for each cell line was separated in a 10% SDS-polyacrylamide gel and immunoblotted with antibodies directed against the indicated receptor. The house keeping gene GAPDH was used for loading control of the lysates. In addition, real-time PCR was performed to evaluate ErbB3 receptor mRNA levels. mRNA was extracted from cells by standard techniques. Real-time PCR was performed as described below, using EGFR and ErbB3 specific primers. Values are expressed as a ratio of EGFR or ErbB3 mRNA: GAPDH mRNA. Levels from PA-1 cells were arbitrarily set to 1.0. Both OVCAR3 and SKOV3 cell lines are good targets for ErbB3 aptamers while OVCAR5 and PA-1 lines, which do not express the Erb B3 receptor, serve as negative controls. In one embodiment, a nanoparticle of the invention is targeted to at least one tumor marker expressed on the surface of a neoplastic cell. In other embodiments, the nanoparticle is targeted to a neoplasm by two or more different aptamers, each of which recognizes a separate tumor marker. In other embodiments, a combination of nanoparticles is provided, each of which comprises a different aptamer recognizing a different tumor marker. Such multiple target therapies facilitate the targeting of a neoplasm containing a diverse population of tumor cells that are heterogeneous in their molecular expression.

### Example 2

### Nanoparticles Encapsulating Photosensitizer

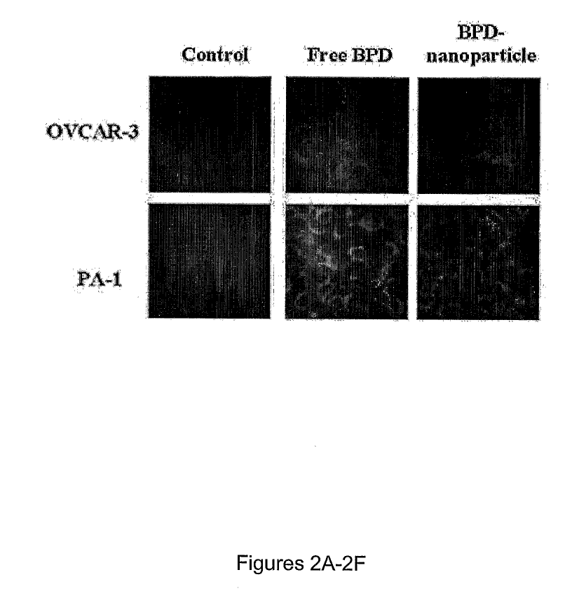

Monodispersed PLGA nanoparticles encapsulating the photosensitizer benzoporphyrin derivative (3PD) (mean diameter of 151 nm), referred to as BPD-PLGA Nanoparticle (BPD-nanoparticles), were generated using the double emulsion solvent evaporation method. See, Liu, Rong; et al., Colloids and Surfaces, B: Biointerfaces (2005) 45(34): 144-153 and Rosca ID, et al., J Control Release. 2004 Sep. 30; 99(2): 271-80. Briefly, PLGA polymer was dissolved in dichloromethane and added to an aqueous solution of BPD. The mixture was subject to sonication and mixed with poly (vinyl alcohol) (PVA) and subject to a second round of sonication. The mixture was stirred at room temperature to allow evaporation of the organic solvents. The resulting nanoparticles were washed and characterized as above. The nanoparticles were incubated with ovarian cancer cell lines, OVCAR3 (FIGS. 2A, 2B, and 2C) or PA-1 (FIGS. 2D, 2E, and 2F). Cells were incubated with media alone, media containing BPD (0.140 uM), or media containing BPD-nanoparticles (0.140 uM BPD equivalent) for three hours. The nanoparticles were approximately 151.1 nm in diameter. Cells were then imaged using a confocal laser scanning microscope with 488 nm excitation and the fluorecence was collected using a 590 nm long pass filter. Fluorescence confocal microscopy of cancer cells showed that the BPD-nanoparticles localized to both target (OVCAR3) and non-target cells (PA-1) (FIG. 2).

### Example 3

### In Vitro Photodynamic Therapy Using BPD-Nanoparticles

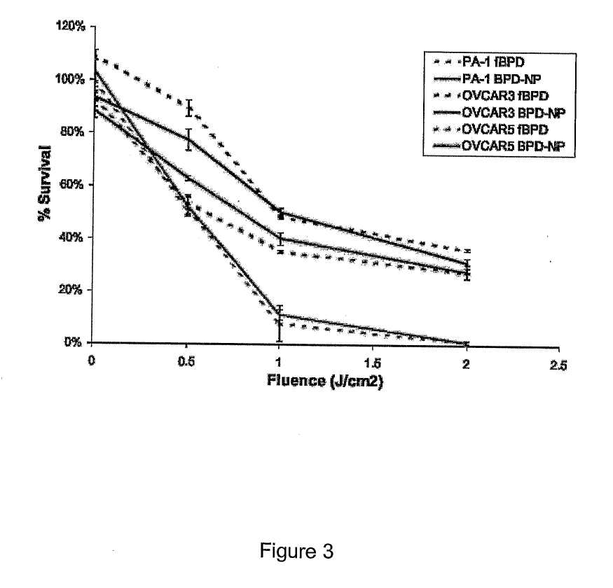

Cells were incubated with 0.140 uM free benzoporphyrin derivative or the equivalent amount of benzoporphyrin derivative in nanoparticles for three hours. After incubation with the BPD-nanoparticles, various cell lines were irradiated with 690 nm light. The cells were then irradiated with varying fluences at 690 nm. Survival was assayed twenty-four hours later using an MTT assay. The survival data reported is relative to untreated cells. As shown in FIG. 3, the BPD-nanoparticles were photochemically active. In addition, the untargeted BPD-nanoparticles were equally cytotoxic in both ErbB3 expressing and non-expressing cell lines which reinforces the need for selective targeting.

### Example 4

### Synthesis of ErbB3 A30 Targeted Aptamer

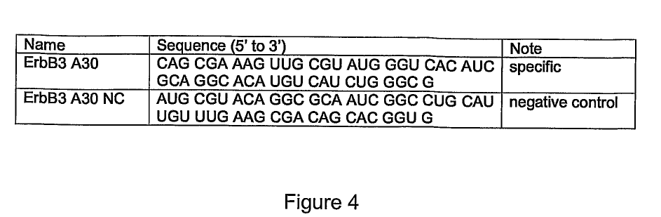

An aptamer that recognizes the ErbB3 receptor was synthesized. The oligonucleotide sequence of the ErbB3-specific aptamer (ErbB3 A30) was obtained from a published article (Chen et al., PNAS, 100(16): 9226-9231, 2003) and the sequence of the negative control aptamer was derived from the randomly scrambled sequence of ErbB3 A30. At the 5′ end, these aptamers were labeled with Fluoroscein for fluorescence microscopy detection in aptamer-cell binding assays. The sequences of the ErbB3-specific aptamer and its control are shown in FIG. 4 (SEQ ID Nos: 1 and 2). The sequence from 5′ to 3′ is listed for both the specific and the scrambled (control) aptamers.

### Example 5

### Selectivity of A30 ErbB3 Aptamer for Target Cells

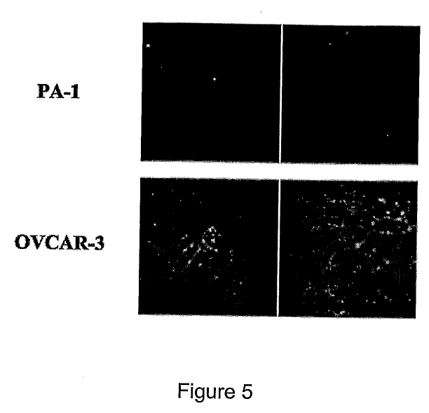

Both ErbB3-expressing cells (OVCAR3) and non-expressing cells (PA-1) were incubated with the ErbB3-specific aptamer and the cells were imaged using a confocal microscope. OVCAR3 (target) and PA-1 (non-target) cells were incubated with 2 uM ErbB3 specific aptamer (tagged on the 5′ end with Fluoroscein) for 15 minutes at 4° C. and then for 20 minutes at 37° C. The cells were then imaged using a confocal laser scanning microscope with 488 nm excitation. The fluorescence was collected using a 515 nm long pass filter. As shown in FIG. 5, the aptamer localized specifically to the OVCAR3 cell line, which expresses the ErbB3 receptor. This demonstrates that the aptamer can selectively target the ErbB3 receptor.

This ErbB3 receptor-specific aptamer is covalently conjugated to a nanoparticle system using an aptamer-nanoparticle conjugation technique. As proof of principle, an aptamer targeting the Prostate Specific Membrane Antigen (PSMA) was conjugated to pegylated nanoparticles encapsulating dextran (Farokhzad et al., Cancer Res,. 64: 7668-7672, 2004). The nanoparticle-aptamer conjugates were then incubated with LNCaP (target) and PC3 (non-target) cells. Using fluorescence microscopy, it was shown that the nanoparticles specifically bound only to the LNCaP cells (Farokhzad et al., Cancer Res, 64: 7668-7672, 2004).

### Example 6

### In Vivo SNACT

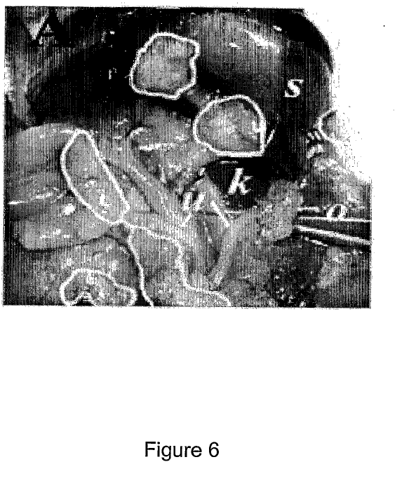

Animal models for human ovarian cancer that mimic clinical disease facilitate the study of nanoparticle therapy in vivo. PDT experiments in a mouse model using OVCAR3 cells have shown that PDT is effective against ovarian cancer. A second model for ovarian cancer is shown in FIG. 6. Swiss Nu/Nu mice were injected intraperitoneally with 31.5×10⁶ SKOV3 and the tumor burden and disease patterns were evaluated twenty-eight days post-innoculation. This model is represents an intraperitoneal resistant human ovarian cancer exhibiting hallmarks of the clinical course in humans. Nanoparticles of the invention and PDT are administered to mouse models of ovarian cancer to characterize the efficacy of such treatment in vivo. The response of the ovarian cancer to the ErbB3 receptor-specific BPD-aptamer therapy is compared to a control. Suitable controls include mice treated with nanoparticles that do not contain BPD nanoparticles

### Example 7

### Quantitation of Photosensitizer Concentration in vivo

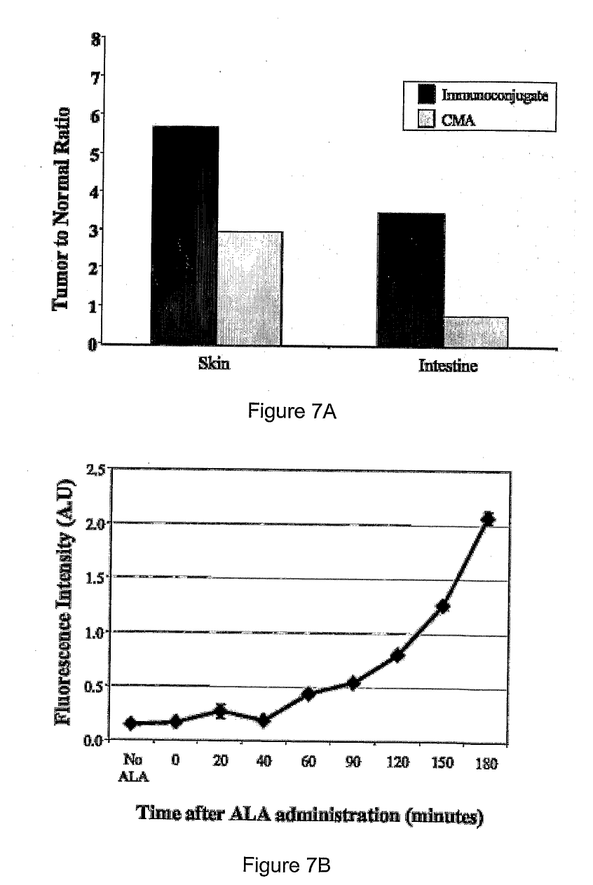

Photosensitizer biodistribution and pharmacokinetics in tissues is characterized using standard extraction and spectroscopy. The photosensitizer chlorin e6 monoethylendiamine monamide (CMA) was measured in various tissues after injection into a mouse as a free form and as an immunoconjugated form. In addition, a fiber-optic based probe dosimeter can be used for on-line in vivo measurement of photosensitizer fluorescence in tissues (FIGS. 7A and B). The biodistribution of CMA or CMA-immunoconjugate was quantified in skin and intestine 24 hours after intraperitoneal injection in tumor-bearing mice. Tissues were harvested and the photosensitizer was extracted and measured spectrophotometrically. FIG. 7B shows the detection of photosensitizer fluorescence in vivo using an on-line fiber optic dosimeter. Photosensitizer was administered intravasculary to a mouse and a fiber optic bundle probe was used to measure photosensitizer concentration as a function of time in the prostate tissue.

### Example 8

### Increased Efficacy of PDT against Ovarian Cancer using Selective Targeting

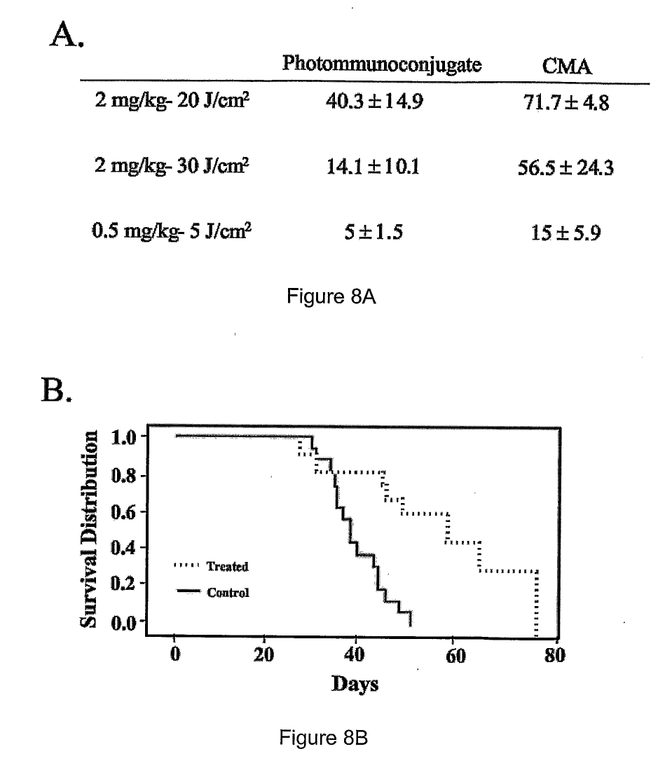

Conjugating targeting moieties, such as aptamers or antibodies, to photosensitizers increases the selectivity of PDT. As proof of this principle, conjugation of CMA to an antibody (OC125) that recognizes an antigen (CA125) that is expressed on many OVCA cells greatly improves the efficacy of the treatment (FIG. 8) as shown in In vivo-in vitro experiments with ascites-bearing mice. Nude mice were injected with 30×10⁶ OVCAR3 cells. After seven days, the mice were subsequently injected with either CMA or CMA conjugated to the monoclonal antibody OC125. Light irradiation was performed twenty-four hours later and the ascities were drained one hour after treatment. Cells were plated and an MTT assay was performed to determine survival. Animals that were given doses of 2 mg/kg were irradiated externally and mice given 0.5 mg/kg were irradiated intraperitoneally. Each group consisted of 7-10 mice and the numbers listed are percentage survival compared to untreated controls FIG. 8B shows Kaplan-Meier survival curves for mice that were treated as described above. The treated mice received four treatments of CMA-immunoconjugate and intraperitoneal light irradiation. As shown in FIGS. 8A and 8B, tumor cell survival decreases and animal survival increases following targeted in vivo PDT.

### Example 9

### Validation of EGFR Targeted PDT by Biomarker Immunohistochemistry

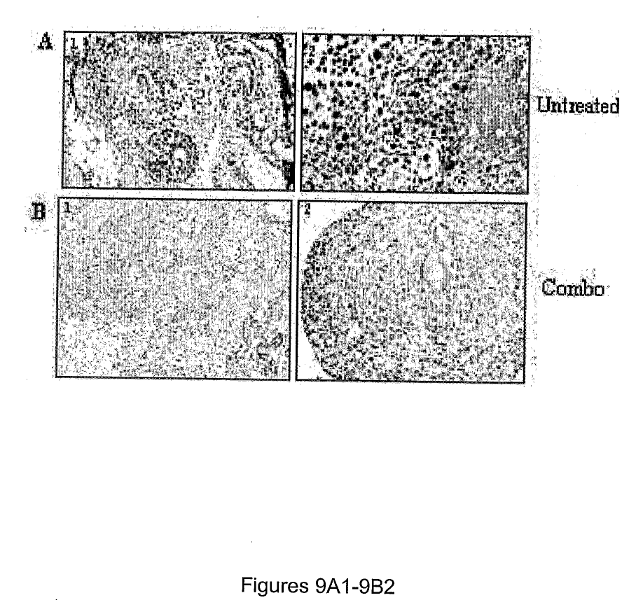

Organs from OVCA animal models are analyzed histopathologically using Proliferating Cell Nuclear Antigen (PCNA) staining. Decreased staining of organs after EGFR-targeted PDT treatment demonstrates that cell proliferation is reduced (FIG. 9) in following treatment with a combination photodynamic therapy and C225 in pelvis (FIG. 9B1) and mesentery (FIG. 9B2), seventy-two hours after the second photodynamic therapy treatment. Staining in untreated control tissues is shown in FIGS. 9B1 and 9B2.

### Example 10

### PhotoSensitizer Nanoparticle Aptamer Conjugate (SNAC) Synthesis

SNACs include a photosensitizer, a biodegradable polymer shell, and a targeting aptamer. These three elements are organized such that the photosensitizer forms the central core of the nanoparticle. The photosensitizer core is encompassed by a biodegradable polymer shell. On the surface of the polymer shell is fixed a targeting aptamer (e.g., ErbB3 receptor-specific aptamer).

In one specific embodiment, 500 ul of the BPD-NP, synthesized as described herein, (˜10 mg/ml in DNase RNase-free water) is incubated with 2000 ml of 400 mmol/L 1-(3-dimethylaminopropyl)-3-ethylcarbodimide hydrochloride (EDC) and 2000 ml of 100 mmol/L N-hydroxysuccinimide (NHS) for 15 minutes at room temperature with gentle stirring. The resulting NHS-activated particles are covalently linked to 500 μl of 3′NH₂ modified ErbB3-specific stabilized aptamer (1 mg/ml in DNase RNase-free water). The resulting SNACs are washed, resuspended, and preserved in suspension form in DNase-free water. The conjugates are subsequently analyzed with spectrophotometry and fluorescence microscopy as described below.

### Example 11

### Physical Characterization of Nanoparticles

Particles are characterized for their sizes, surface charge and surface morphology using Quasi-elastic laser light scattering (QELS), ZetaPALS dynamic light scattering detector, and scanning electron microscopy (SEM). Particles generated from the PEG-PLA block copolymer system can be made in any size range from 75 nm to >10 μm. Nanoparticles were made using the following polymer systems: PLA, PLA-PEG-COOH, and PLGA. These particles had a mean size of 252, 249 and 125 nm, respectively. PLA and PLA-PEG-COOH nanoparticles have a zeta potential (surface charge) of −24 and −50 mV.

### Example 12

### Photophysical Characterization of SNACs

Spectroscopically, the SNAC is characterized for its absorbance and fluorescence. Photophysical characterization relevant for dosimetry/efficacy is used to determine the fluorescence and triplet lifetimes and photobleaching rates of the SNAC in solution and in cells. Such methods are known in the art and are described, for example, in Aveline, B., et al., Photochemistry & Photobiology, 1994. 59(3): p. 328-35, Pogue, B. W., et al., Photochem Photobiol, 1998. 68(6): p. 809-15, Pogue, B. W., Cancer Research, 2001. 61(2): p. 717-24 and Stranadko, E. R et al., Proc SPIE, 2001. 4433: p. 155-157. Briefly, relative fluorescence quantum yields for BPD-NP and for the SNAC samples are calculated from the ratios of the slopes of fluorescence emission intensity versus absorbance plot. Typically 4 to 5 different dilutions of a given sample are prepared in a mix of organic/aqueous solutions with absorbances ranging from 0 to ˜0.1 cm−1 at the excitation wavelength, 428 nm, which is centered near the absorbance peak maximum of the Soret band of BPD. Fluorescence emission intensity is measured as the area under the fluorescence emission peak in the 650 to 800 nm range. Steady-state fluorescence spectra are acquired using a Spex FluoroMax spectrofluorometer (Spex Industries, Inc., Edison, N.J.).

Fluorescence lifetimes are determined by measuring decay signals of BPD-NP and of the various SNAC preparations with varying BPD-NP: ErbB3 A30molar loading ratios using a TimeMaster fluorescence lifetime spectrometer, operated in its StrobeMaster stroboscopic mode (Photon Technology International, Inc., Monmouth Junction, N.J.). The Strobe Master stroboscopic system is based on a technique described by Bonneft, R., et al, Tetrahedron, 2001. 57(591): p. 9513-9547. The excitation source is a N₂ (30%)/He nanosecond lamp.

Samples are prepared in a mix of organic/aqueous solutions and are adjusted by dilution to approximately 0.4 cm−1 at ˜690 nm to ensure that all solutions contain roughly equal amounts of BPD content. Before recording fluorescence decay signals, a BaSO₄ scattering solution is used to measure the nanosecond lamp temporal profile, i.e., the instrument response function (IRF). The experimental fluorescence decays are then acquired with the emission monochromator set at 700 nm. Because sample solutions were excited with the full N₂ (30%)/He lamp spectrum (˜300-400 nm), a long pass filter (>579 nm) is placed in front of the emission monochromator to eliminate second order grating effects. The IRF Instrument Response Factor is then used to fit the experimental fluorescence decay signals by an iterative reconvolution procedure, assuming either a monoexponential or a biexponential free fluorescence decay. The fitting procedure is based on the Marquardt algorithm (Pogue et al., Photochem Photobiol, 68(6): 809-15, 1998; Pogue et al., Cancer Research 61(2): 717-24, 2001; Stranadko et al., Proc SPIE 4433: 155-157, 2001; Oseroff et al., Proc Natl Acad Sci U S A, 83(22): 8744-8, 1986).

Triplet Lifetimes are measured by a pump-probe method (Pogue et al., Cancer Research 61(2): 717-24, 2001). Triplet state behavior in cells is measured in scattering samples using diffuse reflectance laser flash photolysis as described in the literature (Aveline et al., J Photochem Photobiol B, 1995. 30(2-3): 161-9; Aveline et al., J Photochemistry & Photobiology, 59(3): 328-35, 1994; Spikes et al., Journal of Photochemistry & Photobiology. B—Biology, 6(3): 259-74, 1990.; Bonnett et al., Tetrahedron 57(591): 9513-9547, 2001; Losev et al., Proc SPIE 2675: 243-251, 1996; Buczek-Thomas et al., Cell Signal, 10(1): 55-63, 1998; Spikes et al., Journal of Photochemistry & Photobiology. B—Biology, 42(1): 1-11, 1998) Briefly, the remitted light from the front face of a 10×40×1 mm quartz cuvette is monitored at 470 mm using a monochromator with a photomultiplier tube for detection, which used probe light generated from a focused 75-W xenon arc lamp. Laser excitation is provided at 690 nm for BPD-NP from an optical parametric oscillator (Spectra Physics MOPO 710). The resulting change in reflectance of the probe light because of the difference in absorption coefficient between the ground and triplet states is monitored before and after the laser pulse using a high numerical aperture lens to image the front of the cuvette onto the monochromator entrance slit before the detector. The triplet state lifetimes are estimated by a nonlinear Marquardt fitting routine to match the transient reflectance curve to a single exponential decay, with the zero time point fixed to the incident laser pulse.

The photobleaching quantum yield for a given sample solution is calculated as the number of moles of BPD-NP photobleached divided by the number of moles of photons absorbed by the sample solution. Sample solutions include BPD-NP, BPD-NP mixed with unmodified ErbB3 A30, and SNAC. The BPD content of each of the sample solutions is roughly 10 μM, and for the solution of BPD-NP mixed with unmodified ErbB3 A30, the aptamer content is adjusted to be equal to that of the SNAC solution.

Samples are prepared in a mix of organic/aqueous solutions, and all measurements are conducted under air-saturated conditions. Absorption spectra of the sample solutions are measured after various irradiation times, and the number of moles of photodegraded BPD is calculated by monitoring the loss of the 690 nm absorbance peak of BPD. Preliminary studies show that the 514 nm absorbance of the various sample solutions change only very slightly during the photobleaching experiments it can therefore be approximated that the rate of absorbance of 514 nm photons remain constant for each of the sample solutions over the time course of the photobleaching measurements.

### Example 13

### Fluorescence Measurments

One hundred thousand cells are plated on 35 mm-diameter Petri dishes. The cells are seeded in each dish with 3ml growth media and allowed to attach and grow until they form a monolayer that is 60-70% confluent. SNAC and BPD-NP are added to obtain a final BPD concentration of 140 nanoM and incubated with the cells for 3 hours. The solutions are then aspirated from the culture dishes which are washed three times with PBS. The cells are examined with a 63× water immersion objective on the Leica confocal microscope as described Pogue et al., (Cancer Research 61(2): 717-24, 2001). Two-color fluorescence microscopy is used to determine qualitative and semi-quantitative binding of both fluorescein labeled ErbB3 A30 (A30-FITC) and BPD-NP. This will also elucidate the degradation of the ErbB3 A30 from BPD-NP with time in cells.

Cells are prepared for viewing as described, and the SNAC is added and incubated for 1 hour and after a thorough washing the cells are viewed using two separate filter combinations. The first filter has excitation 450-490 nm band pass and emission 514 long pass and is designed for visualizing the FITC fluorescence. The second has excitation 402447 band pass and emission 580 nm long pass and is designed for visualizing BPD-NP fluorescence. There is some overlap between these parameters, i.e. the FITC filter will capture BPD emission but the excitation wavelength is not efficient for BPD-NP. The BPD-NP filter will capture the small tail of the FITC emission and this is very little light. Pictures are taken of the same field using both filters and visual comparisons are made between the two images. The overlap of fluorescence between the two-colored excitations serves to determine the degree to which BPD-NP and ErbB3 A30-FITC fluoresce.

### Example 14

### Binding Specificity

Cells are grown to 90% confluence in twelve well plates and the SNAC is added in serum containing medium. After incubation, the medium is removed and the cells are washed thoroughly with PBS. Trypsin/EDTA is added and the resulting cell suspension is centrifuged. The cell pellet is digested in 0.1 M NaOH; 1% SDS and the fluorescence measured in the SPEX fluorimeter. The digest is then assayed to determine the amount of cell protein by a modified Lowry procedure. The trypsin supernatant is checked for the presence of fluorescence to quantify non-specific binding. Quantitation is achieved using standard solutions of known concentration.

### Example 15

### Preparation and Purification of PLA-PEG Nanoparticle (NP) Encapsulating Chlorin e6 Monoethylene Diamine Disodium Salt (CMA)

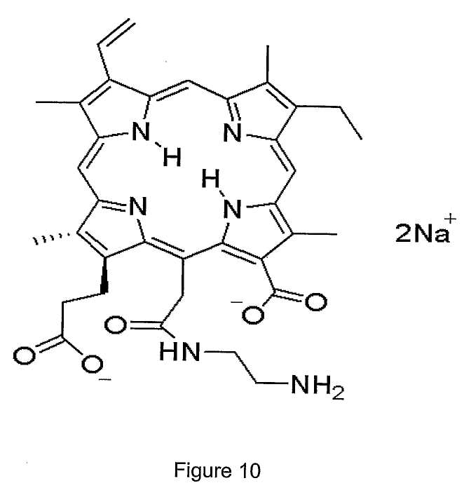

The photosensitizer was purchased from Frontier Scientific, Inc. (Logan, Utah). Its chemical structure is shown in FIG. 10. Heterobifunctional PEG was custom synthesized with hydroxy and carboxyl terminal groups. The PLA was built on the hydroxy end of PEG. Some photophysical properties of the compound related to quantitation and photodynamic therapy have been experimentally explored. In acetonitrile, which is used for lysing CMA-NP to release CMA for regular quantitation using an absorption method, CMA shows an absorption peak at 667 nm in the absorbance spectrum. The CMA content in CMA-NP was periodically checked to insure its stability in the solution. In PBS or culture medium, in which cells are maintained during light irradiation in phototoxicity assays, CMA demonstrated an absorption peak at 655 nm. Thus, a laser source with a wavelength at 670 nm (close to 655 nm) was used for photodynamic therapy. A 1% SDS/0.1 N NaOH solution, was used for lysing cells to release CMA for uptake quantitation by measuring its fluorescence signal. CMA demonstrated an absorption peak at 402 nm. With light at a wavelength of 402 nm for excitation, the emission spectrum for CMA in 1% SDS/0.1 N NaOH was measured. The fluorescence emission peak was at a wavelength of 666 mn. Therefore, 402 nm was used as the excitation wavelength, and 666 nm was used as the emission wavelength in CMA quantitation using fluorescence measurement.

### Example 16

### PLA-PEG Block Copolymer Synthesis

The PLA-PEG block copolymer was synthesized by ring opening polymerization. The nanoparticles were produced by double emulsion (water-in-oil-in-water) to encapsulate the CMA into the polymer matrix during the first emulsion, and then to create the particles in a following emulsion. The CMA was dissolved in water, and the CMA solution was emulsed in the organic phase of dichloromethane, containing the polymer PLA-PEG, by probe sonication. This emulsion was then poured into water with PVA added as a surfactant, and a second sonication produced the nanoparticles. The double emulsed nanoparticles were stirred to evaporate the residual organic solvent, and then collected, purified, and concentrated by ultracentrifugation. The size and surface charge were measured by dynamic light scattering and zeta potential analysis in triplicate, respectively. The encapsulation efficiency and concentration of CMA in the nanoparticles was measured using spectrophotometric measurement of re-dissolved nanoparticle-CMA solution.

### Example 17

### Nanoparticle-Aptamer Conjugation

Fifty microliters of PLA-PEG COOH nanoparticle or microparticle suspension (˜10 μg/μL in DNase RNase-free water) was incubated with 200 μL of 400 mmol/L 1-(3-dimethylaminopropyl)-3-ethylcarbodimide hydrochloride (EDC) and 200 μL of 100 mmol/L N-hydroxysuccinimide (NHS) for 15 minutes at room temperature with gentle stirring. The resulting NHS-activated particles were covalently linked to 50 μL of 3′-NMH2-modified A10 PSMA aptamer (Lupold et al., Cancer Res. 62:4029-33, 2002; 1 μg/μL in DNase RNase-free water) or 3′-NH2 and 5′-FITC-modified A10 PSMA aptamer where indicated. The A10 aptamer has 2′-fluoro-modified ribose on all pyrimidines and a 3′-inverted deoxythymidine cap, which together confer significant nuclease resistance to this molecule. The resulting aptamer-nanoparticle bioconjugates were washed, resuspended, and preserved in suspension form in DNase RNase-free water.

Characterization of the resulting nanoparticles showed that they were in monodispersed form with an average size of 202.0 nm. The surface charge was negative, as a result of the carboxyl end groups of the PLA-PEG copolymer. The zeta potential was ˜30 mV. One mg of CMA was used in the production of the nanoparticles, and the nanoparticles were resuspended in a final volume of 1 ml. A sample of CMA-NP was dissolved in acetonitrile using pure CMA as a standard, and the CMA content in the nanoparticles was determined by measuring the absorbance at a wavelength of 667 nm. The result showed that the concentration of the nanoparticle solution was 0.41 mM or 0.279 mg/ml of equivalent CMA. Therefore, the encapsulation efficiency was 27.9% for this batch of nanoparticle synthesis.

### Example 18

### SNAC Selectively Induced Phototoxicity in Prostate Cancer Cells

Nanaoparticles conjugates were dissolved in 200 ul of PBS, and 5 ul of the stock was diluted in 95 ul of acetonitrile to release the CMA from the carrier (1:20 dilution for this step). 1:100 and 1:1000 dilutions in 1% SDS/0.1N NaOH of the released CMA solution were tested in phototoxicity assays. The total dilutions used were 2,000 and 20,000 fold dilutions. CMA concentration was determined by measuring the diluted stock solution and comparing its fluorescence to the fluorescence of reference standards. 200 ul of stock was diluted into 2 ml medium (1 uM). 1 ml of 1 uM CMA medium was used for imaging analysis and the remaining 1 ml medium was added to 1 ml nanoparticle solution (final 0.5 uM) and used for phototoxicity assays. The nanoparticles were then added to cultured prostate cancer cells. The cells and nanoparticles were incubated together for 90 minutes. The nanoparticles were then activated using light doses of 2, 10, 20 J/cm2 from a diode laser at 665 nm.

The toxicity of SNAC activated with the 10 J/cm2 dose on prostate cancer cell survival was measured. Prostate cells treated with SNAC showed selective phototoxicity relative to control cells that were not targeted for photoxicity by the PSMA aptamer.

A number of embodiments of the invention have been described. Nevertheless, it will be understood that various modifications may be made without departing from the spirit and scope of the invention. Accordingly, other embodiments are within the scope of the following claims.

1. A nanoparticle comprising a polymer shell and one or more aptamers affixed to the shell, wherein the polymer shell contains a photosensitizer core.

2. The nanoparticle of claim 1, wherein the aptamer binds to a marker on the surface of an undesired cell.

3. The nanoparticle of claim 2, wherein the undesired cell is a neoplastic or infected cell.

4. The nanoparticle of claim 2, wherein the aptamer is the ErbB3-specific aptamer of SEQ ID No: 1.

5. The nanoparticle of claim 2, wherein the marker is selected from the group consisting of CA-125, gangliosides G(M2) and G(D3), CD20, CD52, CD33, Ep-CAM, CEA, bombesin-like peptides, prostate specific antigen (PSA), prostate-specific membrane antigen (PSMA), HER2/neu,epidermal growth factor receptor, erbB2, erbB3, erbB4, CD44v6, Ki-67, VEGF, VEGF receptors, VEGFR3, estrogen receptors, Lewis-Y antigen, TGFβ1, IGF-1 receptor, EGFα, c-Kit receptor, transferrin receptor, IL-2R, CO17-1A, tumor-associated antigen MUC1, TGF beta receptor, and a TGF beta receptor of an ErbB family member.

6. The nanoparticle of claim 2, wherein the aptamer binds an ErbB family member selected from the group consisting of EGFR, erbB2, erbB3, and erbB4.

7. The nanoparticle of claim 1, wherein the photosensitizer is a porphyrin.

8. The nanoparticle of claim 1, wherein the photosensitizer is a phenothiazine or a phenoloxazine.

9. The nanoparticle of claim 8, wherein the porphyrin is selected from the group consisting of a porfimer sodium, hematoporphyrin IX, hematoporphyrin ester, dihematoporphyrin ester, synthetic diporphyrin, O-substituted tetraphenyl porphyrin, 3,1-meso tetrakis porphyrin, hydroporphyrin, benzoporphyrin derivative, benzoporphyrin monoacid derivative, monoacid ring derivative, tetracyanoethylene adduct of benzoporphyrin, dimethyl acetylenedicarboxylate adduct of benzoporphyrin, -aminolevulinic acid, benzonaphthoporphyrazine, naturally occurring porphyrin, ALA-induced protoporphyrin IX, synthetic dichlorin, bacteriochlorin tetra(hydroxyphenyl) porphyrin, purpurin, octaethylpurpurin derivative, etiopurpurin, tin-etio-purpurin, porphycene, chlorin, chlorin e6, mono-l-aspartyl derivative of chlorin e6, di-l-aspartyl derivative of chlorin e6, tin(IV) chlorin e6, meta-tetrahydroxyphenylchlorin, chlorin e6 monoethylendiamine monamide, verdin, zinc methyl pyroverdin, copro II verdin trimethyl ester, deuteroverdin methyl ester, pheophorbide derivative, pyropheophorbide, texaphyrin, lutetium (III) texaphyrin, and gadolinium(III) texaphyrin.

10. The nanoparticle of claim 1, wherein the photosensitizer is a photoactive dye.

11. The nanoparticle of claim 11, wherein the photoactive dye is selected from the group consisting of a merocyanine, phthalocyanine, chloroaluminum phthalocyanine, sulfonated aluminum PC, ring-substituted cationic PC, sulfonated AlPc, disulfonated or tetrasulfonated derivative, sulfonated aluminum naphthalocyanine, naphthalocyanine, tetracyanoethylene adduct, nile blue, crystal violet, azure chloride, rose bengal, benzophenothiazinium, phenothiazine derivative and rose bengal methylene blue.

12. The nanoparticle of claim 1, wherein the photosensitizer is selected from the group consisting of a Diels-Alder adduct, dimethyl acetylene dicarboxylate adduct, anthracenedione, anthrapyrazole, aminoanthraquinone, phenoxazine dye, chalcogenapyrylium dye, cationic selena, tellurapyrylium derivative, cationic imminium salt and tetracycline.

13. The nanoparticle of claim 1, wherein the polymer shell is selected from the group consisting of polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt.

14. The nanoparticle of claim 1 wherein the polymer is selected from the group consisting of poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), and poly(ethylene terephthalate).

15. The nanoparticle of claim 1 wherein the polymer is selected from the group consisting of poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate).

16. The nanoparticle of claim 1, wherein the polymer is a PEG-PLGA polymer.

17. The nanoparticle of claim 1, wherein the mass of the nanoparticle is about 1-150 KD.

18. The nanoparticle of claim 1, wherein the mass of the nanoparticle is about 30-60 KD.

19. The nanoparticle of claim 1, wherein the size of the nanoparticle is about 1-500 nm.

20. The nanoparticle of claim 20, wherein the size of the nanoparticle is about 100-400 nm.

21. The nanoparticle of claim 20, wherein the size of the nanoparticle is about 200-300 nm.

22. The nanoparticle of claim 1, wherein the aptamer includes between about 10-100 nucleotides.

23. The nanoparticle of claim 22, wherein the aptamer includes between about 25-75 nucleotides.

24. The nanoparticle of claim 23, wherein the aptamer includes between about 30-60 nucleotides.

25. The nanoparticle of claim 1, wherein the nanoparticle comprises at least two aptamers affixed to the shell, at least one of which binds to an ErbB3 family member.

26. A pharmaceutical composition the composition comprising a therapeutically effective amount of the nanoparticle of claim 1 and a pharmaceutically acceptable excipient.

27-29. (canceled)

30. A method of producing a phototoxic effect in an undesired cell, the method comprising the steps of:
(a) administering the nanoparticle of claim 1 to a cell; and
(b) administering light to the cell in a dose effective to produce a reactive species, thereby producing a phototoxic effect in the cell.

31-39. (canceled)

40. A method of reducing the growth or proliferation of a neoplasm in a subject, the method comprising the steps of:
(a) administering a therapeutically effective amount of the nanoparticle of claim 1 to a subject diagnosed as having a neoplasm; and
(b) administering light to the neoplasm in a dose effective to produce a reactive species, thereby reducing the growth or proliferation of the neoplasm in the subject.

41-46. (canceled)

47. A method of inducing toxicity in a pathogen, the method comprising:
(a) contacting the pathogen with a nanoparticle of claim 1 and
(b) administering light to the pathogen in a dose effective to produce a reactive species, thereby inducing toxicity in the pathogen.

48-51. (canceled)

52. A method of stabilizing, reducing, or ameliorating a pathogen infection in a subject, the method comprising
(a) administering a therapeutically effective amount of a nanoparticle of claim 1 to a subject diagnosed as having a pathogen infection; and
(b) administering light to the site of the infection in a dose effective to produce a reactive species, thereby stabilizing, reducing, or ameliorating the pathogen infection in the subject.

53-66. (canceled)

67. A kit for producing a phototoxic effect in an undesired cell comprising a nanoparticle of claim 1 and instructions for use thereof.

68-69. (canceled)

70. A packaged pharmaceutical comprising:
a) a nanoparticle of claim 1, and
b) instructions for using said nanoparticle to produce a phototoxic effect in an unwanted cell.

71-73. (canceled)


SEQUENCE LISTING (2 sequences)

SEQ ID NO: 1 - RNA, 49 nt, Artificial Sequence
  Description of Artificial Sequence Synthetic oligonucleotide
cagcgaaaguugcguaugggucacaucgcaggcacaugucaucugggcg

SEQ ID NO: 2 - RNA, 49 nt, Artificial Sequence
  Description of Artificial Sequence Synthetic oligonucleotide
augcguacaggcgcaaucggccugcauuguuugaagcgacagcacggug